(19) United States
(12) Patent Application Publication
(10) Pub. No.: US 2003/0008307 A1
(43) Pub. Date: Jan. 9, 2003

(54) Methods for diagnosing activated protein C resistance associated with a factor V genetic mutation and compositions thereof

(75) Inventors: John H. Griffin, Del Mar, CA (US); Judith Greengard, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 10/115,563

(22) Filed: Apr. 2, 2002

Related U.S. Application Data
(63) Continuation of application No. 08/410,488, filed on Mar. 24, 1995, now abandoned.

(51) Int. Cl. C12Q 1/68; C07H 21/04; C12P 19/34

(52) U.S. Cl. 435/6; 435/91.2; 536/23.5

(57) ABSTRACT

A method for identifying a Factor V gene mutation resulting in activated protein C resistance comprising detecting in a nucleic acid sample isolated from a human the presence of a genetic mutation characterized as a change from a guanine nucleotide to an adenine nucleotide at nucleotide position 205 in exon 10 of the Factor V gene that is associated with replacement of arginine 506 by glutamine, thereby identifying said mutation.

### GOVERNMENT SUPPORT

This invention was made with the support of the United States Government and the United States Government has certain rights in the invention pursuant to the National Institutes of Health Contracts HL31950 AND HL21544.

### TECHNICAL FIELD

The present invention relates to a method for detecting a coagulation Factor V allele resulting in activated Protein C (APC) resistance in a human. This allele is a point mutation characterized as a change from a guanine nucleotide to an adenine nucleotide at nucleotide position 205 in exon 10 of the normal Factor V gene. The exon 10 point mutation corresponds to nucleotide position 1691 of the Factor V cDNA sequence.

### BACKGROUND OF THE INVENTION

Blood coagulation reactions and thrombosis play major roles in cardiovascular diseases. Risk factors for these diseases include both inherited and acquired risk factors. Studies of hereditary thrombophilia, defined as an increased tendency towards venous thrombotic disease in relatively young adults, provide insights into factors that regulate thrombosis.

Hereditary thrombophilia has been associated with molecular defects of antithrombotic factors, including antithrombin III, protein C and protein S. However, molecular defects which involve these factors have been identifiable in only 10-15% of thrombophilic patients. Therefore, a molecular defect has not been identified in the vast majority of these patients (Gladson et al, Thromb. Haemost. 59:18, 1988; Allaart et al, Lancet 341:134, 1993; Horellou et al, Br. Med. J. 289:1285, 1984; Pabinger et al, Thromb. Res. Suppl. VI 136a, 1986; Malm et al, Thromb. Haemost. 68:7, 1992; Ben-Tal et al, Thromb. Haemost. 61:50, 1989).

Blood coagulation is a complex process in which a cascade of zymogen activations results in the formation of thrombin and the subsequent conversion of fibrinogen to fibrin (Furie et al, Cell 33:505, 1988; Davie et al, Biochem. 30:10363, 1991). The rate of coagulation is regulated by positive and negative feedback loops. In an example of a positive feedback loop, the coagulation process is greatly accelerated when thrombin activates Factors V and VIII to form Factors Va and VIIIa (Mann et al, Annu. Rev. Biochem. 57:915, 1988; Kane et al, Blood 71:539, 1988). Factors Va and VIIIa, when bound to phospholipid on platelets and endothelial cells or in phospholipid vesicles, are involved in the further activation of prothrombin and Factor X, respectively. In an example of a negative feedback loop, protein C (E.C. 3.4.21.69) becomes activated in the presence of catalytic amounts of thrombin to form activated protein C (APC). APC then inactivates Factors Va and VIIIa in the presence of Ca²⁺ and phospholipid by proteolytic cleavage (Marlar et al, Blood 59:1067, 1982; Mann et al, Annu. Rev. Biochem. 57:915, 1988; Esmon et al, J. Biol. Chem. 264:4743, 1989). Inactivation of Factors Va and VIIIa by APC thereby inhibits the coagulation process.

Recently a syndrome, termed activated protein C (APC) resistance, was described in 20-50% of the venous thrombophilic patients in which a molecular defect has not been identified (Dahlback et al, Proc. Natl. Acad. Sci. USA 90:1004, 1993; Griffin et al, Blood 82:1989, 1993; Svensson et al, N. Enal. J. Med. 330:517, 1994; Koster et al, Lancet 342:1503, 1993; Faioni et al, Thromb. Haemost. 70:1067, 1993). APC resistance is characterized as a poor anticoagulant response to APC in plasma (Dahlback et al, Proc. Natl. Acad. Sci. USA 90:1004, 1993) and is an autosomal dominant trait which cosegregates with risk of thrombosis in affected families (Svensson et al, New Engl. J. Med. 330:517, 1994).

APC resistance has been routinely assessed by comparing the activated partial thromboplastin times (APTT) determined in the absence and presence of APC for thrombophilic patients to results for control subjects without a familial history of thrombophilic events (Dahlback et al, Proc. Natl. Acad. Sci. USA 90:1004-1008, 1993). APC prolongs the APTT in normal plasma by inactivating Factors Va and VIIIa via specific proteolytic cleavage as further described herein. The APC resistance assay is based upon the fact that the addition of APC to a clotting test such as the APTT normally causes an increase (prolongation) in the APTT, whereas in an APC resistant patient's plasma the APC-induced prolongation is significantly reduced compared to that observed in normal plasma.

In the APTT assay, an APC resistance ratio, sometimes termed an APC sensitivity ratio, is determined as the clotting time of test plasma in the presence of APC and CaCl₂ divided by the clotting time of test plasma in the presence of CaCl₂. For test plasmas under certain typical conditions, APC resistance ratios of ≧2.19 are considered to be normal in males and ≧1.94 in females. The normal range depends on a variety of assay variables.

Additionally, APTT assays have been used to assess whether the APC resistance of thrombophilic patients' plasmas is caused by abnormalities in either of the APC substrates, Factors V and VIII. In these assays, either the patient's plasma or normal plasma is combined with either Factor V-deficient plasma, or Factor VIII-deficient plasma and the effect of the APC on the combination of plasma on the APTT assay is determined. In patients whose APC resistance is due to an abnormality in Factor V, combining the patient's plasma with Factor V-deficient plasma gives a mixture that exhibits resistance to APC in comparison to the mixtures of normal plasma with Factor V-deficient plasma. However, in patients whose APC resistance is due to an abnormality in the V substrate, combining the patient's plasma with Factor VIII-deficient plasma has the same result on the APC resistance tests as combining the Factor VIII-deficient plasma with normal plasma (Sun et al, Blood 83:3120, 1994).

Alternatively, APC resistance due to an abnormality in Factor V has also been assessed in the APTT assay by the addition of purified normal or APC resistant patient's Factor V to Factor V-deficient plasma followed by APC resistance testing (Sun et al, Blood 83:3120, 1994).

While the APTT assay is valuable in the assessment of clotting times, obtaining consistent and reproducible results can be affected by several variables. These variables include the administration of oral anticoagulants such as warfarin or heparin to patients (Svensson et al, N. Engl. J. Med. 33:517, 1994), activation of platelets during the preparation of platelet poor plasma (Cooper et al, Br. J. Haematol. 86 (suppl. 1): 33 (abstr), 1994), and the use of frozen and thawed plasma (Girolami et al, Lancet 343:1288 (letter), 1994; Jones et al, Br. J. Haematol. 86 (suppl. 1): 32 (abstr), 1994). Since thrombophilic patients are often treated with oral anticoagulants, (e.g. warfarin), therefore, the APC resistance of these patients cannot be accurately assessed with the standard APTT assay (Dahlback et al, Proc. Natl. Acad. Sci USA 90:1004, 1993).

An alternative approach for assaying APC resistance resulting from a defect in Factor Va in these patients, such as that described in the present invention, is therefore highly desirable.

Factor V is a glycoprotein with a molecular weight (Mr) of 330 kilodaltons (kDa). The Factor V protein is a single polypeptide which consists of an amino terminal heavy chain region, a connecting region, and a carboxy terminal light chain region. When Factor V is cleaved by thrombin at three arginine amino acid residues at amino acid positions 709, 1018, and 1545 (Kane et al, Blood 71:539, 1988; Suzuki et al, J. Biol. Chem. 257:6556, 1982; Esmon, J. Biol. Chem. 254:964, 1979; Mann et al, Annu. Rev. Biochem. 57:915, 1988) during the coagulation process to form the activated state of Factor V (Va), the connecting region is removed from between the heavy and light chain regions and the resultant two polypeptides consisting of the heavy and light chains are held together by Ca²⁺ ions (Kane et al, Biochem. 26:6508, 1987).

Proteolytic inactivation of formed Factor Va by APC normally requires binding of Factor Va to a membrane surface and sequential cleavage of the Factor Va polypeptide. The first cleavage is following the amino acid residue arginine at position 506 in the heavy chain and the second cleavage is following amino acid residue arginine at position 306 in the heavy chain (Kalafatis et al, Blood 82:58a, 1993; Kalafatis et al, J. Biol. Chem. 268:27246, 1993 and J. Biol. Chem. 269:31869, 1994). The inactivation process may require the binding of APC to a high-affinity APC binding site in the Factor Va light chain (Krishnaswamy et al, J. Biol. Chem. 261:9684, 1986; Walker et al, J. Biol. Chem. 265:1484, 1990).

The Factor Va light chain binds to phospholipid with high affinity in a Ca²⁺-independent manner (Kane et al, Blood 71:539, 1988; Mann et al, Annu. Rev. Biochem. 57:915, 1988) and associates tightly with APC on phospholipid surfaces, e.g., the dissociation constant (Kd) for bovine APC to Factor Va:phospholipid is approximately 7 nanomoles/liter (Krishnaswamy et al, J. Biol. Chem. 261:9684, 1986). The presence of the phospholipid phosphotidylethanolamine in vesicles has been shown to enhance APC anticoagulant activity and APC inactivation of purified Factor Va (Smirnov et al, J. Biol. Chem. 269:816, 1994). The importance of high-affinity binding of APC to Factor Va was demonstrated in recent studies that showed an active site mutant of protein C, in which the amino acid residue serine was substituted with the amino acid residue alanine at position 360, lacked hydrolytic activity and exhibited approximately 20% anticoagulant activity in clotting assays and in prothrombinase assays (Sun et al, Blood 82:148a, 1993).

Previous investigation examined the effect of the posttranslational modifications sulfation and phosphorylation on the physiological activity of Factor V. Kalafatis et al hypothesized that sulfation of Factor V affected the interaction between thrombin and Factor V thereby affecting the cleavage of Factor V by thrombin to form Factor Va (Kalafatis et al, Blood 81:704, 1993 and 91: 1396, 1994). However, in APC resistance patients, cleavage of Factor V by thrombin to form Factor Va is comparable to that of normal patients and therefore is not the cause of APC resistance. Hortin determined that partially phosphorylated Factor Va was inactivated at a higher rate than native Factor Va and suggested that the susceptibility of Factor Va to APC cleavage is probably altered by phosphorylation of the Serine at amino acid residue position 690 of the Factor Va heavy chain (Hortin, Blood 76:946, 1990).

While posttranslational modifications such as sulfation and phosphorylation may affect the interactions of Factor Va with other serum components as described above, these modifications are not the cause of APC resistance in the instant invention.

It was also hypothesized that a defect in an uncharacterized cofactor for APC which copurified with Factor V, designated APC cofactor 2, was responsible for the APC resistance and that APC resistance was caused by a selective defect in the anticoagulant properties of Factor V (Dahlback et al, Proc. Natl. Acad. Sci. USA 90:1004, 1993). In fact, Dahlback et al stated that Factor Va lacked APC cofactor activity because thrombin treatment of normal Factor V destroyed the APC cofactor 2 activity of Factor Va and that they had previously ruled out the possibility that their APC resistant patients Factor Va was APC resistant (Dahlback et al, Proc. Natl. Acad. Sci. USA 91:1396, 1994).

Sun et al described the partial purification of Factor Va from two patients with APC resistance syndrome and demonstrated that purified Factor Va protein was resistant to inactivation by APC (Sun et al, Blood 83:3120-3125, 1994).

While these two independent reports suggest that Factor V may either serve as a cofactor which is abnormal or that Factor Va may be abnormal in APC resistance patients, each report could also be explained by abnormalities in the molecule(s) which associate with Factor V in plasma or, as suggested by Kalafatis and Hortin, posttranslational modification(s) of Factor Va.

The present invention now resolves the molecular basis for APC resistance that has up to this time been unresolved in view of the above-described assays and patient analyses. The genetic basis for APC resistance as determined by the methods of this invention resides in a single point mutation in the Factor V gene, the result of which retards the normal inactivation of Factor Va by APC.

### BRIEF SUMMARY OF THE INVENTION

The present invention relates to methods, diagnostic systems and compositions useful for detecting a new coagulation Factor V mutation resulting in resistance to activated Protein C, (APC), in a human. A point mutation characterized as a change from a guanine nucleotide to an adenine nucleotide occurs at nucleotide position 205 in exon 10 of the Factor V gene. The corresponding position of the point mutation in the cDNA derived from the Factor V gene is at nucleotide position 1691.

Thus, in one embodiment, a human genetic screening method is contemplated. The method comprises assaying a nucleic acid sample isolated from a human for the presence of a Factor V gene point mutation characterized as a change of a guanine nucleotide to an adenine nucleotide at nucleotide position 205 in exon 10 of Factor V gene. The method also allows for the determination of a patient's genotype through the analysis of the alleles comprising the Factor V gene.

In a preferred embodiment, the method comprises treating, under amplification conditions, a sample of genomic DNA from a human with a polymerase chain reaction (PCR) primer pair for amplifying a region of human genomic DNA containing nucleotide position 205 in exon 10 of Factor V gene. The PCR treatment produces an amplification product containing the requisite nucleotide position 205 of exon 10, which is then assayed for the presence of a guanine nucleotide change to an adenine nucleotide at this nucleotide position.

In one aspect of screening genomic DNA from a patient to determine the presence or absence of the above-described point mutation in the alleles comprising the Factor V gene, a PCR primer pair is utilized in a PCR amplification where the first primer hybridizes to a noncoding strand of Factor V gene at a location 3′ to the point mutation at nucleotide position 205 in the noncoding strand and a second primer hybridizes to the coding strand of Factor V gene at a location 3′ to this position in the coding strand.

In this method, the PCR primer pair produces an amplification product containing a restriction endonuclease site if the point mutation is not present in the amplified DNA. In one embodiment, assaying of the genomic DNA amplification products for the absence of the point mutation is accomplished by nucleotide sequencing.

In another embodiment, assaying of the resultant amplification products involves treatment, under restriction conditions, with a restriction endonuclease that recognizes the restriction site and cleaves the amplification product if the point mutation is absent. As a result, the restriction digestion patterns between a normal allele of a Factor V gene and an affected allele allows for the determination of the presence or absence of the point mutation at nucleotide position 205 in exon 10 in a Factor V gene allele in a patient.

With the primer pairs of this invention, a preferred amplified nucleotide region of the Factor V gene of a normal allele lacking the point mutation contains the nucleotide sequence corresponding to 5′-ACAGGCGAGG-3′ (SEQ ID NO 6), or a fragment thereof. The presence of the guanine nucleotide corresponding to nucleotide position 205 in exon 10 confers a Mnl I restriction endonuclease digestion site present in a normal allele.

The corresponding preferred amplified nucleotide region of the Factor V gene of an affected allele contains the new point mutation as underlined within the nucleotide sequence corresponding to 5′-ACAGGCLAGG-3′ (SEQ ID NO 7), or a fragment thereof. The presence of the adenine nucleotide corresponding to nucleotide position 205 in exon 10 destroys a Mnl I restriction endonuclease digestion site present in a normal allele.

A preferred PCR primer pair for amplifying the nucleotide region containing nucleotide position 205 in exon 10 has a first and second primer with the respective nucleotide sequences 5′-CATACTACAGTGACGTGGAC-3′ (SEQ ID NO 4) and 5′-TGTTCTCTTGAAGGAAATGC-3′ (SEQ ID NO 5). The primer pair provides for the amplification of a region of a Factor V gene from either normal or affected alleles. In a normal allele, the resultant amplified nucleotide region consists essentially of a nucleotide sequence shown in SEQ ID NO 2 having a guanine nucleotide at nucleotide position 205 of exon 10. In an affected allele containing the point mutation, the amplified nucleotide region consists essentially of a nucleotide sequence shown in SEQ ID NO 3 having an adenine nucleotide at nucleotide position 205 of exon 10. The presence of the latter destroys a normal Mnl I restriction endonuclease site.

Therefore, the presence of the point mutation destroys the ability of the restriction endonuclease Mnl I to digest the Factor V DNA in the amplified nucleotide region. As a result, the differential Mnl I restriction digestion patterns between a normal allele and an affected allele allows for the determination of the presence of the point mutation in a Factor V gene allele in a patient having APC resistance.

In another aspect of screening genomic DNA from a patient, an alternative PCR primer pair of a first and second primer is utilized to produce an amplification product containing a restriction endonuclease site if the point mutation is present. Assaying for the presence of the point mutation includes nucleotide sequencing, restriction digestion analysis and the like.

A resultant preferred amplified nucleotide region of the Factor V gene of an affected allele having the point mutation contains the nucleotide sequence 5′-AAGCTT-3′ (SEQ ID NO 23). The corresponding preferred amplified nucleotide region of the Factor V gene of a normal allele that lacks the point mutation contains the nucleotide sequence 5′-GAGCTT-3′ (SEQ ID NO 25).

A preferred first and second primer of a primer pair for amplifying the above-described nucleotide sequences in normal and affected alleles have the respective nucleotide sequences 5′-CATACTACAGTGACGTGGAC-3′ (SEQ ID NO 4) and 5′-TTACTTCAAGGACAAAATACCTGTAAAGCT-3′ (SEQ ID NO 24). A nucleotide region, consisting essentially of the sequence shown in SEQ ID NO 19, of such an amplified mutant allele having an adenine nucleotide at nucleotide position 205 in exon 10 generates a Hind III restriction endonuclease digestion site. The corresponding nucleotide region, consisting essentially of the sequence shown in SEQ ID NO 18, amplified from a normal allele having a guanine nucleotide does not contain the requisite Hind III site. Therefore, differential Hind III restriction digestion patterns of genomic DNA amplified with the primer pairs of SEQ ID NOs 4 and 24 provide an alternative method for determining the presence or absence of the point mutation from genomic DNA of a patient.

In a further embodiment, a method for screening messenger RNA (mRNA) from a human for identifying a genetic mutation at nucleotide position 1691 in a Factor V cDNA is contemplated. The method comprises isolation of mRNA from a human and synthesis of a complementary strand of DNA (cDNA) followed by PCR with a primer pair for amplifying a region of human cDNA containing nucleotide position 1691 of Factor V cDNA. The resultant amplified cDNA product is then assayed for the presence of a change from a guanine nucleotide to an adenine nucleotide at cDNA nucleotide position 1691 to identify the mutation.

As with amplifying genomic DNA, to amplify cDNA a PCR primer pair is utilized in a PCR amplification where the first primer hybridizes to a noncoding strand of Factor V cDNA at a location 3′ to the point mutation at nucleotide position 205 of the noncoding strand and a second primer hybridizes to a coding strand of Factor V cDNA at a location 3′ to this position in the coding strand.

In this method, the PCR primer pair produces a cDNA amplification product containing a restriction endonuclease site if the point mutation is not present. In one embodiment, the resultant cDNA amplification product is assayed by nucleotide sequencing to determine the absence of the point mutation.

In another embodiment, the resultant cDNA amplification products are then treated, under restriction conditions, with a restriction endonuclease that recognizes the restriction site and cleaves the cDNA amplification product if the point mutation is absent. As a result, the restriction digestion pattern difference between a normal allele and an affected allele allows for the determination of the presence or absence of the point mutation in cDNA.

With the primer pairs for amplifying cDNA, a preferred amplified nucleotide region of the Factor V gene of a normal allele lacking the point mutation contains the nucleotide sequence corresponding to 5′-ACAGGCGAGG-3′ (SEQ ID NO 6), or a fragment thereof. As with genomic DNA, the presence of the guanine nucleotide corresponding to nucleotide position 1691 in the cDNA confers a Mnl I restriction endonuclease digestion site present in a normal allele.

The corresponding preferred amplified mutant nucleotide region of cDNA contains the nucleotide sequence corresponding to 5′-ACAGGCAAGG-3′ (SEQ ID NO 7), or a fragment thereof. The presence of the adenine nucleotide corresponding to nucleotide position 1691 in the cDNA destroys a Mnl I restriction endonuclease digestion site present in normal cDNA.

A preferred PCR primer pair for amplifying the nucleotide region containing nucleotide position 1691 in Factor V cDNA has a first and second primer with the respective nucleotide sequences 5′-CAGGAAAGGAAGCATGTTCC-3′ (SEQ ID NO 10) and 5′-TGCCATTCTCCAGAGCTAGG-3′ (SEQ ID NO 11). The primer pair provides for the amplification of a nucleotide region from either normal or affected cDNA. In normal cDNA, the resultant amplified nucleotide region consists essentially of a nucleotide sequence shown in SEQ ID NO 27 having a guanine nucleotide at nucleotide position 1614 in SEQ ID NO 27 which, corresponds to nucleotide position 1691 in Factor V cDNA. The basis for the discrepancy in the nucleotide position of the guanine nucleotide being at 1614 in SEQ ID NO 27 as compared to 1691 in intact Factor V cDNA stems from the convention for numbering the amplified cDNA as shown in SEQ ID NO 27. In the latter, nucleotide position 1 corresponds to nucleotide position 78 in Factor V cDNA as shown in SEQ ID NO 13 and in the both FIGS. 6A and 6B.

In cDNA containing the point mutation, the amplified nucleotide region consists essentially of a nucleotide sequence shown in SEQ ID NO 28 having an adenine nucleotide at nucleotide position 1614, that corresponds to 1691 in intact Factor V cDNA.

The normal and mutant amplified cDNA products respectively listed in SEQ ID NOs 27 and 28 are preferred products for assaying by nucleotide sequencing to determine the absence or presence of the guanine to adenine nucleotide point mutation.

Another preferred PCR primer pair for amplifying the nucleotide region containing nucleotide position 1691 in Factor V cDNA has a first and second primer with the respective nucleotide sequences 5′-CATACTACAGTGACGTGGAC-3′ (SEQ ID NO 4) and 5′-TGCTGTTCGATGTCTGCTGC-3′ (SEQ ID NO 12). The primer pair provides for the amplification of a 124 base pair nucleotide region from either normal or affected cDNA. In normal cDNA, the resultant amplified nucleotide region consists essentially of a nucleotide sequence shown in SEQ ID NO 13, from nucleotide position 1601 to nucleotide position 1724, having a guanine nucleotide at cDNA nucleotide position 1691. In cDNA containing the point mutation, the amplified nucleotide region consists essentially of a nucleotide sequence shown in SEQ ID NO 26, from nucleotide position 1601 to nucleotide position 1724, having an adenine nucleotide at that same position.

The presence of the adenine nucleotide at that site destroys a normal Mnl I restriction endonuclease site. As such, the normal and mutant amplified cDNA products respectively listed in SEQ ID NOs 13 and 26, both from nucleotide position 1601 to 1724, are preferred products for assaying by Mnl I restriction digestion to determine the absence or presence of the guanine to adenine nucleotide point mutation. Therefore, the presence of the point mutation destroys the ability of the Mnl I restriction endonuclease to digest the Factor V cDNA in the amplified nucleotide region.

Another preferred cDNA-amplified nucleotide region produced by the methods of this invention having an adenine nucleotide at nucleotide position 1691 is the complete Factor V cDNA sequence shown in SEQ ID NO 26, from nucleotide position 9 to nucleotide position 6917.

As a result, as with the methods of this invention for screening genomic DNA, both nucleotide sequence analysis and the differential Mnl I restriction digestion patterns between a normal cDNA and an affected cDNA allows for the determination of the presence of the point mutation in mRNA from a patient having APC resistance.

Diagnostic kits useful for the detection of a genetic mutation in a Factor V gene at nucleotide position 205 of exon 10 associated with activated Protein C resistance in a patient genomic DNA or mRNA nucleic acid sample are also contemplated. The kit comprises, in an amount sufficient to perform at least one assay, a pair of primers comprising a first primer and a second primer capable of producing by PCR an amplification product that contains nucleotide position 205 in exon 10 of the Factor V gene.

In one aspect of the diagnostic kit, primers are in separate containers. Preferred primer pairs include a first primer and a second primer for amplifying either genomic DNA or cDNA. Particularly preferred genomic DNA primer pairs are the paired nucleotide sequences listed in SEQ ID NOs 4 and 5, and SEQ ID NOs 4 and 24. Particularly preferred cDNA primer pairs are the paired nucleotide sequences listed in SEQ ID NOs 10 and 11, and SEQ ID NOs 4 and 12.

In a further embodiment, the diagnostic kit further comprises a control polynucleotide sequence derived from a normal Factor V gene having a nucleotide sequence shown in SEQ ID NOs 2, 18, 13 (from nucleotide position 1601 to 1724) and 27.

The diagnostic kit of this invention further comprises a control polynucleotide sequence derived from a mutant Factor V gene having a genetic mutation at nucleotide position 205 in exon 10. Preferred control mutated polynucleotide sequences include those shown in SEQ ID NOs 3, 19, 26 (from nucleotide position 1601 to 1724) and 28.

The present invention also provides compositions of isolated polynucleotide sequences derived from a Factor V gene having a genetic mutation at nucleotide position 205 in exon 10. In a preferred embodiment, the isolated polynucleotide sequence comprises a nucleotide sequence from about 10 nucleotides to 6909 nucleotides in length. Preferred polynucleotide sequences include those containing nucleotide sequences shown in SEQ ID NOs 7 and 23, or fragments thereof.

Also contemplated are compositions of preferred polynucleotide sequences consisting essentially of a genomic DNA nucleotide sequences shown in SEQ ID NOs 3, 19, along with cDNA nucleotide sequences shown in SEQ ID NOs 28 and 26, the latter being a large fragment from nucleotide position 9 to 6917 and also a smaller fragment from nucleotide position 1601 to 1724. SEQ ID NO 26 contains the Factor V cDNA sequence that contains an adenine at nucleotide position 1691.

Another contemplated composition of the present invention is a polynucleotide primer comprising a nucleotide sequence shown in SEQ ID NO 24 from nucleotide position 26 to nucleotide position 31. The preferred primer is capable of producing an amplification product containing a Hind III restriction endonuclease site in a Factor V gene having a guanine to adenine point mutation at nucleotide position 205 of exon 10. A preferred polynucleotide primer consists essentially of a nucleotide sequence shown in SEQ ID NO 24.

### BRIEF DESCRIPTION OF THE DRAWINGS

FIG. 1, presented over FIGS. 1A-1C, gives the nucleotide sequence (SEQ ID NO 13) and corresponding amino acid residue sequence (SEQ ID NO 14) of Factor V cDNA. The complete cDNA nucleotide and encoded amino acid residue sequence is provided for comparison to the nucleotide sequence derived from the cDNA of APC resistance patients as described in Example 1D. The amino acid residue labeled as “1” corresponds to the amino terminal amino acid residue of the plasma protein. The deduced amino acid residue sequence of Factor V consists of 2224 amino acid residues that comprises a 28 amino acid residue leader peptide, a 709 amino acid residue heavy chain region, a 836 amino acid residue connecting region, and a 650 amino acid residue light chain region. The heavy vertical downward arrow represents the leader peptide cleavage site; the filled circles represent potential N-linked glycosylation sites and the curved arrows represent thrombin cleavage sites.

The amino acid residue sequence determined by amino acid sequencing is indicated by the solid overbar for amino acid residue sequence obtained from human Factor V and a dashed overbar for amino acid residue sequence obtained from bovine Factor V.

The nucleotide sequence 5′-GAATTCCG-3′ (SEQ ID NO 13, from nucleotide position 1 to nucleotide position 8) at the beginning of the nucleotide sequence corresponds to the linker sequence inserted during preparation of the cDNA library and therefore is not a part of the Factor V cDNA. However, by convention, the nucleotides corresponding to the nucleotide sequence of the linker are included in the cDNA sequence when nucleotide positions are given (Jenny et al, Proc. Natl. Acad. Sci. USA 84:4846, 1987). Thus, the linker sequence corresponds to nucleotide positions 1 to 8. For the remaining indicated nucleotide positions, refer to SEQ ID NO 13.

Immediately following the nucleotide sequence of the linker are 82 nucleotides of the 5′ untranslated region of the Factor V cDNA from nucleotide positions 9 to 90. Following the nucleotide sequence of the 5′ untranslated region are the 84 nucleotides encoding the leader peptide of the Factor V cDNA from nucleotide positions 91 to 174. Following the nucleotide sequence of the leader peptide are the 2127 nucleotides encoding the heavy chain region of the Factor V cDNA from nucleotide positions 175 to 2301.

Following the nucleotide sequence of the heavy chain region are the 2508 nucleotides encoding the connecting region of the Factor V cDNA from nucleotide positions 2302 to 4809. Following the nucleotide sequence of the connecting region are the 1950 nucleotides encoding the light chain region of Factor V cDNA from nucleotide positions 4810 to 6762. Following the nucleotide sequence of the light chain region are 163 nucleotides of the 3′ untranslated region of the Factor V cDNA from nucleotide positions 6763 to 6925. The 3′ untranslated region includes a putative polyadenylation signal sequence 5′-AATAAA-3′ (SEQ ID NO 13, from nucleotide position 6893 to nucleotide position 6898) located 12 nucleotides upstream of the poly(A) tail. The poly(A) tail is indicated by the nucleotide sequence 5′-AAAAAAA-3′ from nucleotide positions 6911 to 6917 (SEQ ID NO 13). Following the poly(A) tail is the linker sequence 5′-CGGAATTC-3′ from nucleotide positions 6918 to 6925 (SEQ ID NO 13). Both the 5′ and 3′ linker nucleotide sequence were inserted during preparation of the cDNA library and therefore are not part of the Factor V cDNA.

FIGS. 2A and 2B respectively show a portion of the nucleotide sequences of normal (SEQ ID NO 2) and mutant (SEQ ID NO 3) alleles of exon 10 and intron 10 of the Factor V gene. The coding strand of the double-stranded DNA produced by PCR amplification of the Factor V gene from genomic DNA with primers FV7 (SEQ ID NO 4) and FVINT102 (SEQ ID NO 5) as described in Example 1B is shown in both FIGS. 2A and 2B. The nucleotide sequence in upper and lower case letters represents the nucleotide sequence of exon 10 and intron 10, respectively. The numbering above the upper case nucleotide sequence corresponds to the nucleotide position in exon 10 of the Factor V gene. The numbering above the lower case nucleotide sequence corresponds to the nucleotide position in intron 10 of the Factor V gene.

The double underlined nucleotide sequence in FIG. 2A represents one of two Mnl I restriction endonuclease sites in exon 10 of the Factor V gene and was cleaved to verify that the DNA fragment could be cleaved in the presence of the restriction endonuclease Mnl I as described in Example 1B.

The double underlined Mnl I restriction site is also referred to as the 5′ Mnl I site as it is located 5′ on the coding strand to the other Mnl I site containing nucleotide position 205 of exon 10.

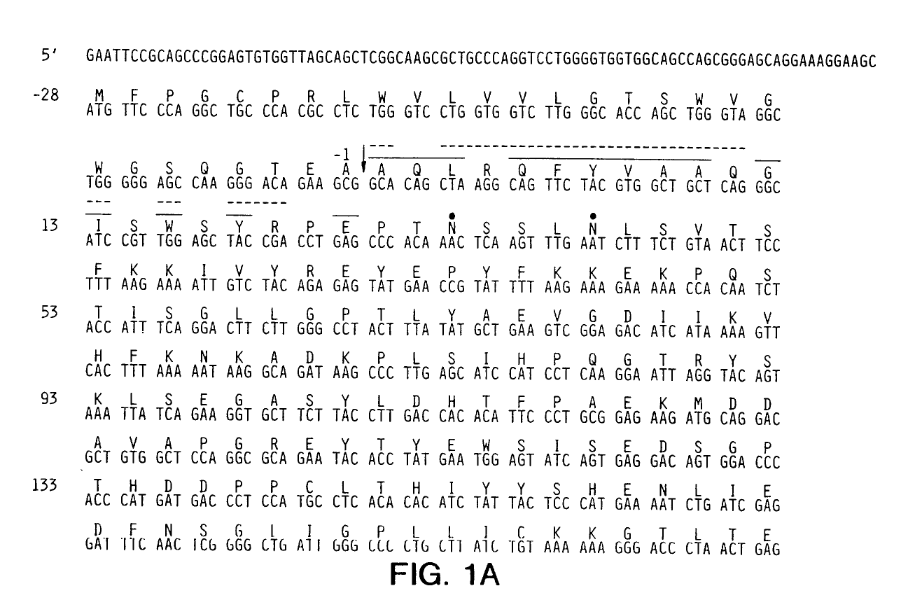

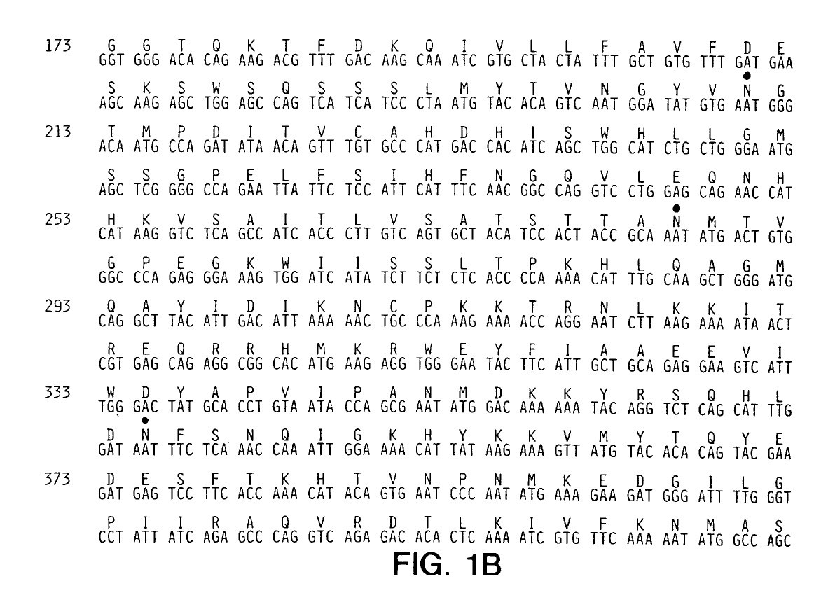

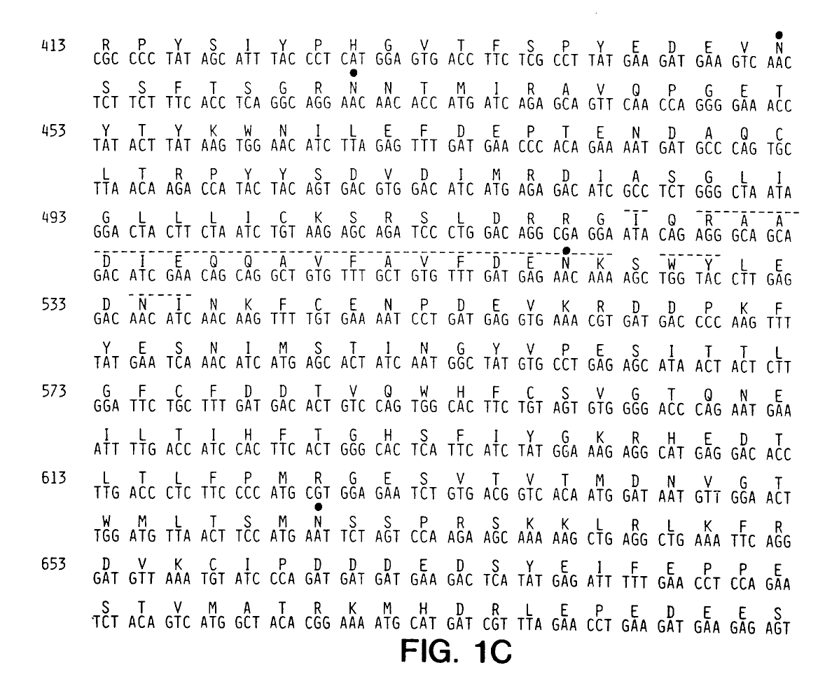

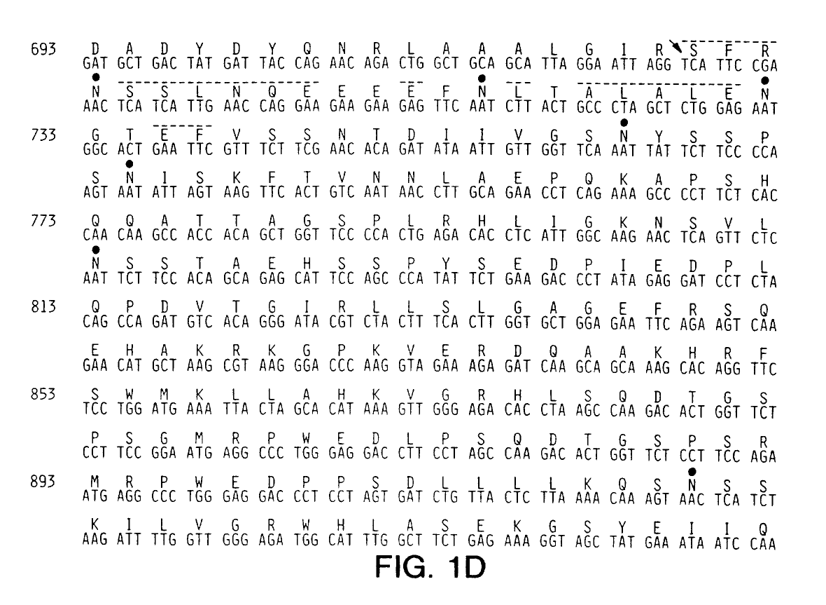

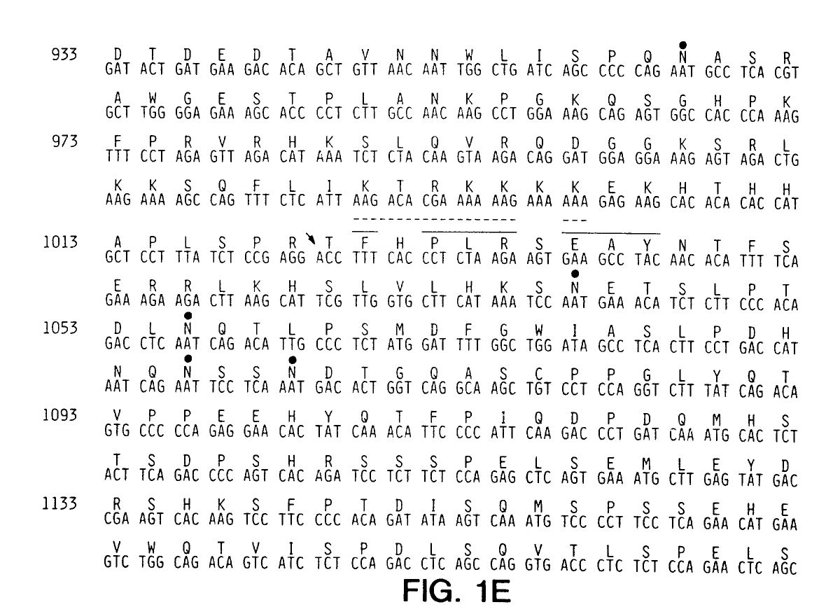

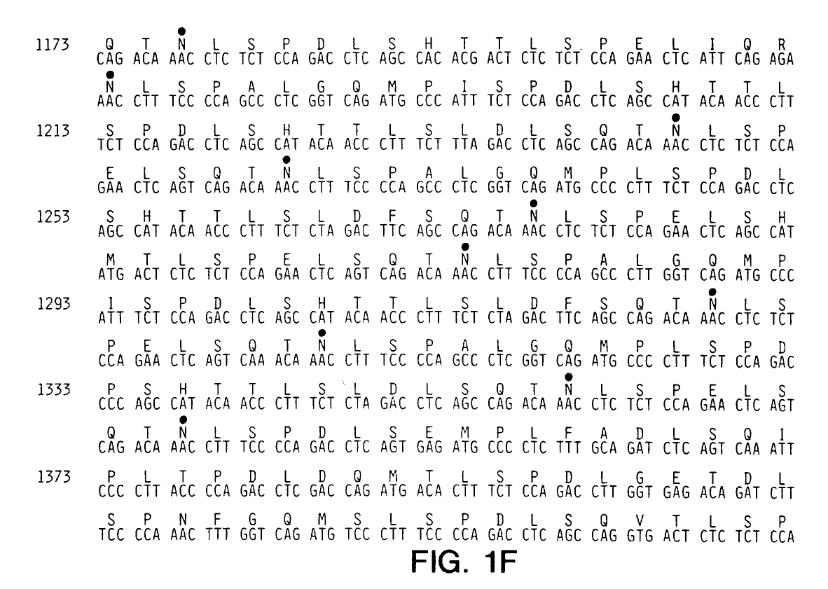

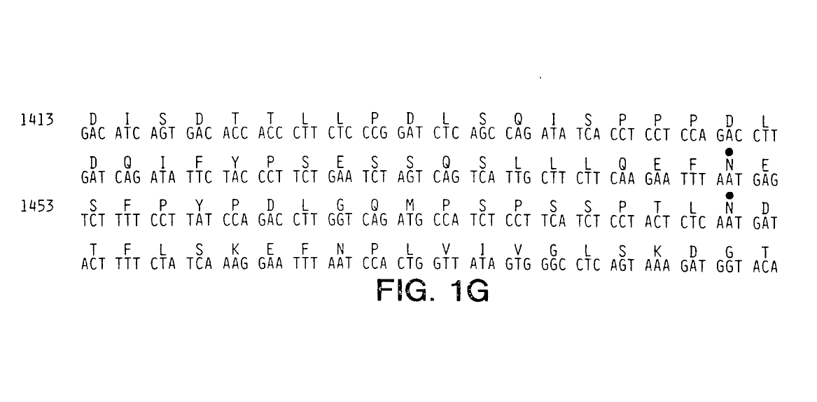

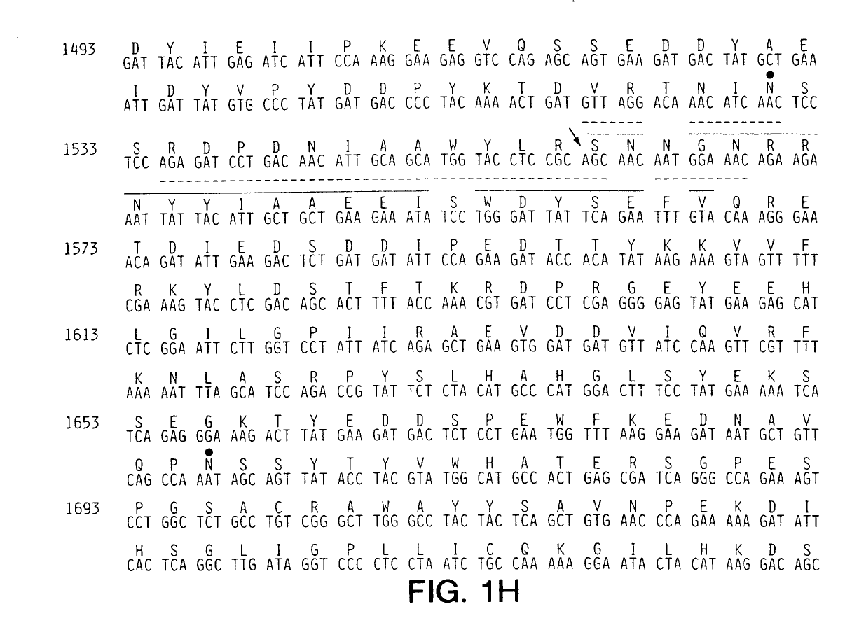

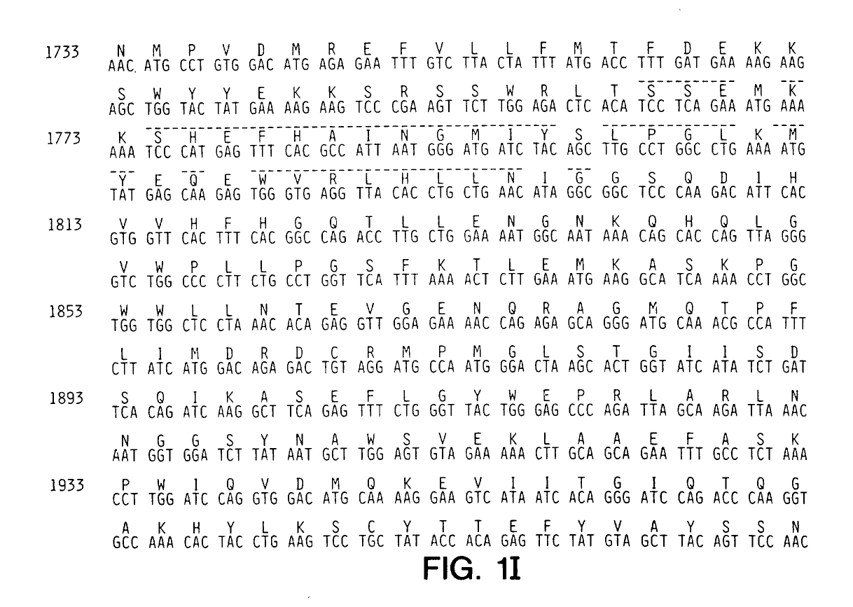

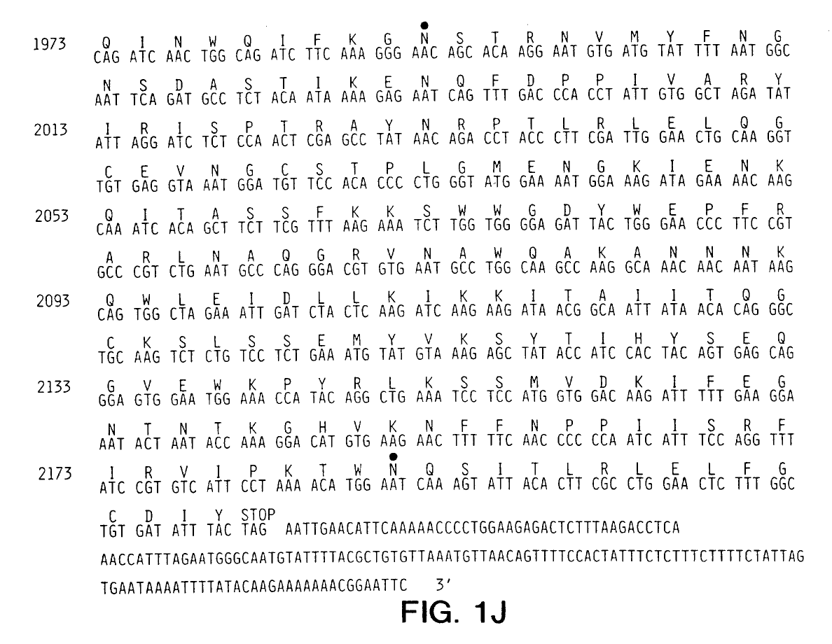

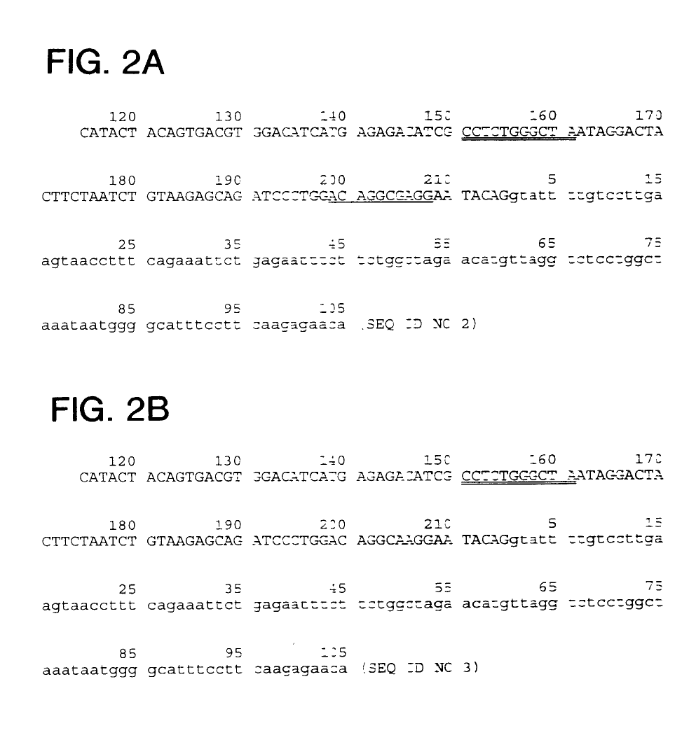

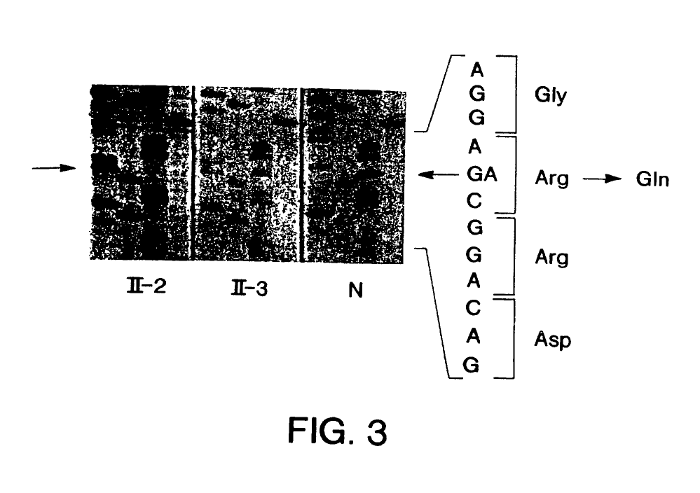

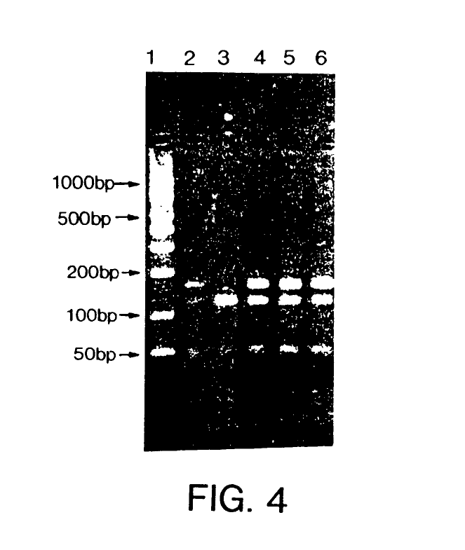

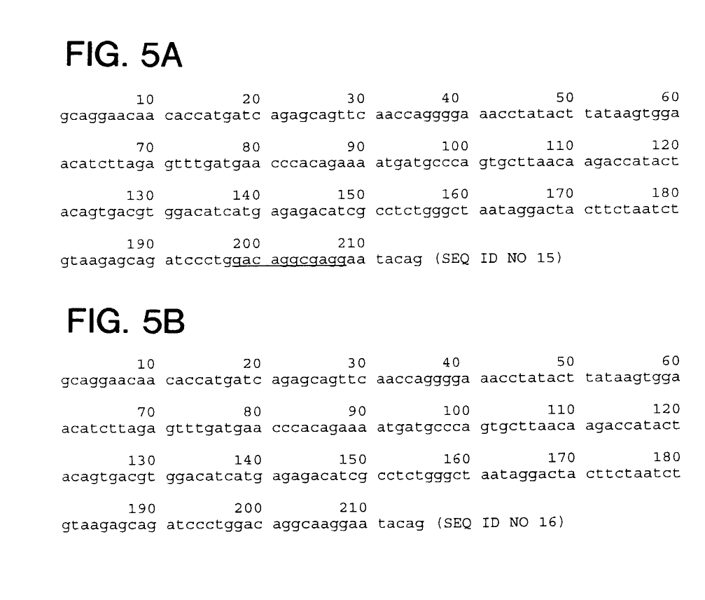

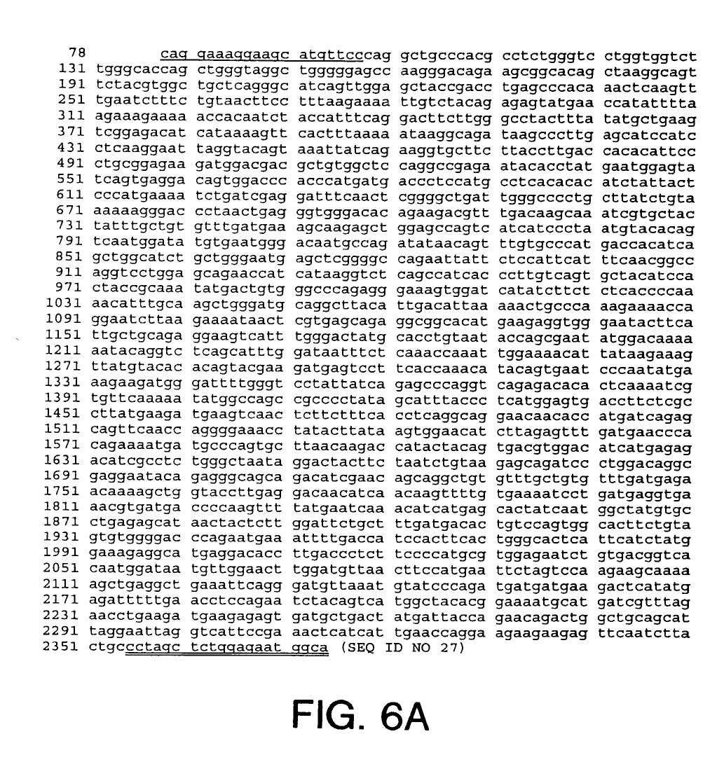

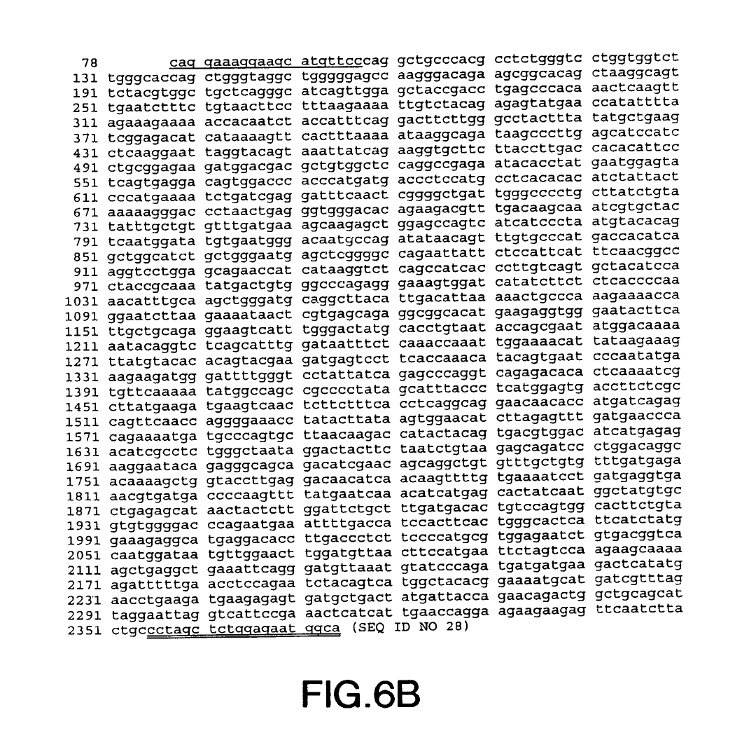

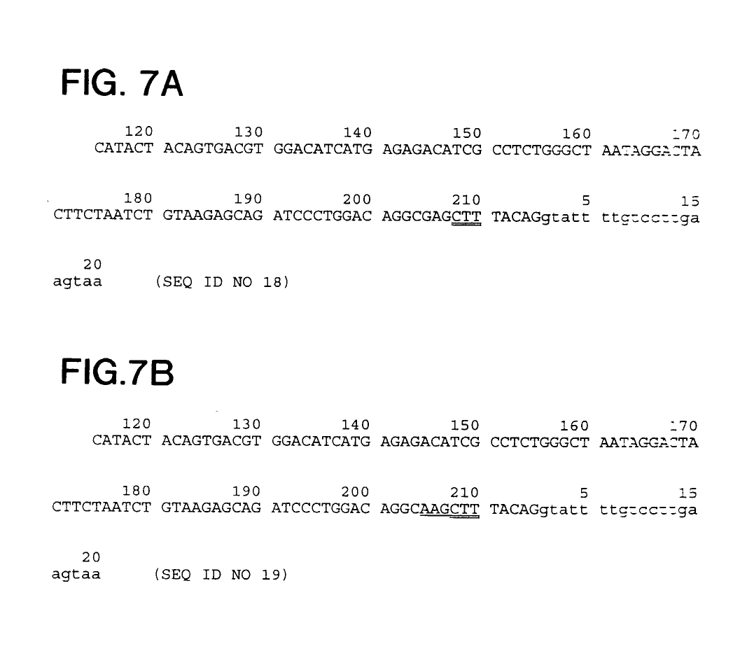

The single underlined nucleotide sequence in FIG. 2A represents the Mnl I restriction endonuclease site containing the guanine nucleotide at position 205 in exon 10 of the Factor V gene (SEQ ID NO 15) which corresponds to position 1691 of the Factor V cDNA sequence as shown in FIG. 1C and in SEQ ID NO 13.

The single underlined Mnl I restriction site is also referred to as the 3′ Mnl I site as it is located 3′ on the coding strand to the 5′ Mnl I site.

The same nucleotide position in FIG. 2B contains an adenine nucleotide indicating the point mutation in exon 10 of the Factor V gene (SEQ ID NO 3), and thereby lacking the Mnl I restriction site present in the normal allele.

FIG. 3 represents the partial autoradiographic nucleotide sequence of normal and mutant alleles of the Factor V cDNA as determined in Example 1D. The samples shown are a homozygous daughter (II-2), a heterozygous son (II-3), and a normal control (N). The nucleotide sequence from the three different samples is given in each of four lanes from left to right as adenosine, cytosine, guanine, and thymine. The four lanes of each sample, II-2, II-3, and N are separated from each other by the solid bar. The arrow at the left indicates the position of nucleotide 1691 of the Factor V cDNA nucleotide sequence. The nucleotide sequence given at the right is from 5′ at the bottom to 3′ at the top of the figure and represents the cDNA coding sequence of Factor V cDNA from nucleotide positions 1684 to 1695. The presence of the nucleotides guanine and adenine (GA) indicate the nucleotide of the normal and mutant alleles at cDNA nucleotide position 1691, respectively. The amino acid residues given at the right are from the amino terminus at the bottom to the carboxy terminus at the top and represent the amino acid residue sequence encoded by the nucleotides. A change in the codon from CGA to CAA corresponding to cDNA nucleotide positions 1690 to 1692 results in a change in the amino acid residue encoded by the triplet from an arginine (Arg) to a glutamine (Gln).

FIG. 4 is a photograph of an agarose gel containing DNA representing a portion of genomic DNA including nucleotide position 205 in exon 10 of the Factor V gene which has been incubated in the presence of the restriction endonuclease Mnl I and separated electrophoretically as described in Example 1B. Lane 1 contains DNA molecular weight markers as indicated in base pairs (bp). Lanes 2, 4, 5, and 6 contain DNA isolated from APC resistance patients that are heterozygous for the point mutation at nucleotide position 205 in exon 10 of the Factor V gene. Lane 3 contains DNA isolated from a normal patient that is homozygous for the normal or nonmutant allele.

FIGS. 5A and 5B give the respective nucleotide sequence representing the normal (SEQ ID NO 15) and mutant (SEQ ID NO 16) alleles of exon 10 of the Factor V gene. The numbering given is from the first nucleotide in exon 10 of the Factor V gene following intron 9 of the genomic DNA to the last nucleotide of exon 10 prior to intron 10 (Kane et al, Biochem. 26:6508, 1987). In FIG. 5A, the single underlined sequence represents the Mnl I site in the normal allele in exon 10 of the Factor V gene. FIG. 5B does not contain a Mnl I site due to the change in the nucleotide at position 205 in exon 10 of the Factor V gene from a guanine to an adenine.

FIGS. 6A and 6B are the respective normal (SEQ ID NO 27) and mutant (SEQ ID NO 28) nucleotide sequences of the coding strand of the amplification product of Factor V cDNA with primers FV13 (SEQ ID NO 10) and FV2 (SEQ ID NO 11) as described in Example 1D. The single underlined nucleotide sequence corresponds to the FV13 primer. The double underlined nucleotide sequence corresponds to the inverse complement of the FV2 primer.

The numbering along the left side of the figure corresponds to the nucleotide position in the cDNA of Factor V as described in Jenny et al, Proc. Natl. Acad. Sci. USA 84:4846, 1987. The cDNA PCR product contains 13 nucleotides of the 5′ untranslated sequence from nucleotide positions 78 to 90, 84 nucleotides encoding the Factor V leader sequence from nucleotide positions 91 to 175, the nucleotide sequence encoding the Factor V heavy chain from nucleotide positions 176 to 2130, and 73 nucleotides of the connecting sequences from nucleotide positions 2131 to 2204.

The amplified normal and mutant cDNA nucleotide sequences are listed respectively in SEQ ID NOs 27 and 28 where position 78 of each FIGS. 6A and 6B are now listed as nucleotide position 1 in the Sequence Listing. As a result, the guanine to adenine point mutation in Factor V cDNA at nucleotide position 1691 occurs in SEQ ID NOs 27 and 28 at nucleotide position 1614. Similarly, nucleotide position 2374 in FIGS. 6A and 6B is now nucleotide position 2297 in the Sequence Listing.

In addition, in SEQ ID NO 17, where the heavy chain amplified cDNA beginning with Factor V cDNA nucleotide 78 is shown with a “N” in position 1614 (corresponding to cDNA position 1691), the N is either a guanine in a normal Factor V cDNA and an adenine in a mutant Factor V cDNA.

FIGS. 7A and 7B respectively show the coding strand nucleotide sequence of the amplification products derived from a Factor V gene of a normal (SEQ ID NO 18) or mutant (SEQ ID NO 19) allele by PCR amplification with primers FV7 and FV506tst2 as described in Example 1C.

In both figures, the nucleotide sequence in upper and lower case letters represents the nucleotide sequence of exon 10 and intron 10, respectively. The numbering above the upper case nucleotide sequence corresponds to the nucleotide position in exon 10 of the Factor V gene. The numbering above the lower case nucleotide sequence corresponds to the nucleotide position in intron 10 of the Factor V gene.

The double underlined nucleotide sequence in FIGS. 7A and 7B indicates point mutations that are introduced at nucleotide positions 208-210 of exon 10 into the amplification product by PCR amplification with primers FV7 and FV506tst2 as described in Example 1C. The single underlined nucleotide sequence in FIG. 7B represents the Hind III restriction endonuclease site containing the adenine nucleotide point mutation at nucleotide position 205 in exon 10 of the Factor V gene (SEQ ID NO 19). The same nucleotide position in FIG. 7A contains a guanine nucleotide indicating the absence of a point mutation at nucleotide position 205 in exon 10 of the Factor V gene (SEQ ID NO 18).

### DETAILED DESCRIPTION OF THE INVENTION

A. Definitions

TABLE OF CORRESPONDENCE

| Code Group | | Nucleotide(s) |
|---|---|---|
| A | A | adenine |
| C | C | cytosine |
| G | G | guanine |
| T | T | thymine (in DNA) |
| U | U | uracil (in RNA) |
| Y | C or T(U) | pyrimidine |
| R | A or G | purine |
| M | A or C | amino |
| K | G or T(U) | keto |
| S | G or C | strong interaction (3 hydrogen bonds) |
| W | A or T(U) | weak interaction (2 hydrogen bonds) |
| H | A or C or T(U) | not-G |
| B | G or T(U) or C | not-A |
| V | G or C or A | not-T or not-U |
| D | G or A or T(U) | not-C |
| N | G,A,C or T(U) | any |

Allele: A variant of DNA sequence of a specific gene. In diploid cells a maximum of two alleles will be present, each in the same relative position or locus on homologous chromosomes of the chromosome set. When alleles at any one locus are identical, the individual is said to be homozygous for that locus. When the alleles differ, the individual is said to be heterozygous for that locus. Since different alleles of any one gene may vary by only a single base, the possible number of alleles for any one gene is very large. When alleles differ, one is often dominant to the other. The allele which is not dominant is said to be recessive. Dominance is a property of the phenotype and does not imply inactivation of the recessive allele by the dominant allele. In numerous examples the normally functioning (wild-type) allele is dominant to all mutant alleles of more or less defective function. In such cases the general explanation is that one functional allele out of two is sufficient to produce enough active gene product to support normal development of the organism (i.e., there is normally a two-fold safety margin in quantity of gene product). The mutant allele may or may not result in the defective function of the gene which it encodes. If the mutant allele does not result in the defective function of the gene which it encodes, it may be termed a carrier state. If the mutant allele results in the defective function of the gene which it encodes and results in a an increased risk of a disease state, it may be termed a “disease” allele or risk factor allele.

Nucleotide: A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence”, and their grammatical equivalents, and is represented herein by a sequence whose left to right orientation is in the conventional direction of 5′-terminus to 3′-terminus.

Base Pair (bp): A partnership of adenine (A) with thymine (T), or of cytosine (C) with guanine (G) in a double stranded DNA molecule. In RNA, uracil (U) is substituted for thymine.

Nucleic Acid: A polymer of nucleotides, either single or double stranded.

Polynucleotide: A polymer of single or double stranded nucleotides. As used herein “polynucleotide” and its grammatical equivalents will include the full range of nucleic acids. A polynucleotide will typically refer to a nucleic acid molecule comprised of a linear strand of two or more deoxyribonucleotides and/or ribonucleotides. The exact size will depend on many factors, which in turn depends on the ultimate conditions of use, as is well known in the art. The polynucleotides of the present invention include primers, probes, RNA/DNA segments, oligonucleotides or “oligos” (relatively short polynucleotides), genes, vectors, plasmids, and the like.

Gene: A nucleic acid whose nucleotide sequence codes for an RNA or polypeptide. A gene can be either RNA or DNA.

Duplex DNA: A double-stranded nucleic acid molecule comprising two strands of substantially complementary polynucleotides held together by one or more hydrogen bonds between each of the complementary bases present in a base pair of the duplex. Because the nucleotides that form a base pair can be either a ribonucleotide base or a deoxyribonucleotide base, the phrase “duplex DNA” refers to either a DNA-DNA duplex comprising two DNA strands (ds DNA), or an RNA-DNA duplex comprising one DNA and one RNA strand.

Complementary Bases: Nucleotides that normally pair up when DNA or RNA adopts a double stranded configuration.

Complementary Nucleotide Sequence: A sequence of nucleotides in a single-stranded molecule of DNA or RNA that is sufficiently complementary to that on another single strand to specifically hybridize to it with consequent hydrogen bonding.

Conserved: A nucleotide sequence is conserved with respect to a preselected (reference) sequence if it non-randomly hybridizes to an exact complement of the preselected sequence.

Hybridization: The pairing of substantially complementary nucleotide sequences (strands of nucleic acid) to form a duplex or heteroduplex by the establishment of hydrogen bonds between complementary base pairs. It is a specific, i.e. non-random, interaction between two complementary polynucleotides that can be competitively inhibited.

Nucleotide Analog: A purine or pyrimidine nucleotide that differs structurally from A, T, G, C, or U, but is sufficiently similar to substitute for the normal nucleotide in a nucleic acid molecule.

Upstream: In the direction opposite to the direction of DNA transcription, and therefore going from 5′ to 3′ on the noncoding strand, or 3′ to 5′ on the RNA transcript.

Downstream: Further along a DNA sequence in the direction of sequence transcription or read out, that is, traveling in a 3′- to 5′-direction along the noncoding strand of the DNA or 5′- to 3′-direction along the RNA transcript.

Stop Codon: Any of three codons that do not code for an amino acid, but instead cause termination of protein synthesis. They are UAG, UAA and UGA and are also referred to as a nonsense, termination, or translational stop codon.

Reading Frame: Particular sequence of contiguous nucleotide triplets (codons) employed in translation. The reading frame depends on the location of the translation initiation codon.

B. Diagnostic Methods

The present invention provides a novel method for screening humans for Factor V alleles comprising the Factor V gene to determine a patient's genetic basis for APC resistance. The invention was born out of the discovery that APC resistance can be caused by a mutation in the Factor V gene DNA sequence wherein the guanine nucleotide at position 205 in exon 10 of the Factor V gene has been substituted by an adenine nucleotide.

Portions of the genomic sequence of the normal wild type Factor V gene have been determined. The gene is comprised of 25 exons and 24 introns and spans greater than 80 kilobases of genomic DNA (Cripe et al, Biochem. 31:3777, 1992).

The nucleotide sequence of a normal exon 10 sequence having 215 base pairs is shown in FIG. 5A and is also listed in SEQ ID NO 15. As transcribed and translated, the triplet codon from nucleotide position 204-206 that includes the guanine nucleotide of nucleotide position 205, CGA, in a normal Factor V gene encodes an arginine amino acid residue.

Nucleotide position 205 in exon 10 has been determined to correspond to nucleotide position 1691 of the complementary DNA (cDNA) nucleotide sequence as described by Jenny et al, Proc. Natl. Acad. Sci. USA 84:4846, (1987) and shown both in FIGS. 1A-1C and in SEQ ID NO 13.

The cDNA sequence of Factor V (SEQ ID NO 13) comprises a 6672 base pair (bp) coding region, a 90 bp 5′ untranslated region, and a 163 bp 3′ untranslated region (Jenny et al, Proc. Natl. Acad. Sci. USA 84:4846, 1987; FIGS. 1A-1C). The encoded amino acid residues sequence contains 2224 amino acids (SEQ ID NO 14) that includes a 28 amino acid residue leader peptide. The numbering of the nucleotides and amino acid residues of Factor V as referred to herein is according to the numbering given in Jenny et al, Proc. Natl. Acad. Sci. USA 84:4846, 1987.

The cDNA nucleotide sequence as given by Jenny et al includes a 5′ 8-mer nucleotide sequence, 5′-GAATTCCG-3′, (SEQ ID NO 13 from nucleotide position 1 to nucleotide position 8)and a second 8-mer at the 3′ end, 5′-CGGAATTC-3′ (SEQ ID NO 13 from nucleotide position 6918 to nucleotide position 6925), both of which are not present in the gene sequence. The 8-mer sequences are Eco RI linkers used to construct the cDNA library. Therefore, the actual length of the cDNA sequence is not 6925 as shown in FIGS. 1A-1C and SEQ ID NO 13 but rather is 6909 base pairs in length.

As defined for use in this invention, a normal or unaffected (wild type) Factor V gene does not have a point mutation at nucleotide position 205 in exon 10. In other words, a normal Factor V gene of this invention contains a guanine nucleotide at nucleotide position 205 in exon 10.

As defined for use in this invention, the counterpart to a normal Factor V gene is a mutant or affected Factor V gene having a point mutation at nucleotide position 205 in exon 10. In other words, a mutant Factor V gene of this invention contains an adenine nucleotide at nucleotide position 205 in exon 10 rather the normal guanine nucleotide. The nucleotide sequence of the described mutant exon 10 sequence having 215 base pairs is shown in FIG. 5B and is also listed in SEQ ID NO 16. As transcribed and translated, the triplet codon from nucleotide position 204-206 that includes the adenine nucleotide of nucleotide postion 205, CAA, in a mutant Factor V gene encodes a glutamine amino acid residue.

Therefore, the genetic mutation present in a Factor V gene characterized as a point mutation from a guanine to an adenine is referred to as a Factor V gene mutation.

A Factor V gene is a nucleic acid whose nucleotide sequence encodes either a normal Factor V protein or mutant Factor V protein. The nucleic acid can be in the form of genomic DNA, mRNA or cDNA, and in single or double stranded form.

The assay methods of this invention are useful for screening a patient's nucleic acids to determine the presence or absence of the point mutation at nucleotide position 205 of exon 10 that is associated with APC resistance. The methods provide the ability to distinguish between Factor V alleles that are homozygous normal, homozygous mutant or heterozygous. In other words, the methods as described herein allow for the distinction between a patient having two mutated alleles comprising a Factor V gene, a patient have only one mutated allele while the other allele is normal, and a patient having two normal alleles.

Thus, as more fully described in Section B2 and B3 below, the methods of this invention generally involves preparing a nucleic acid sample for screening and then assaying the amplified products for the guanine to adenine point mutation in alleles that comprise a Factor V gene.

In preferred embodiments, the nucleic acid sample is enriched for the presence of Factor V allelic material. Enrichment is typically accomplished by subjecting the genomic DNA or mRNA to a primer extension reaction employing a polynucleotide synthesis primer as described herein. Particularly preferred methods for producing a sample to be assayed use preselected polynucleotides as primers, a general description of which is provided below in Section Bla. The primers are used in a polymerase chain reaction (PCR), a general description of which is provided below in Section Blb, to form an amplified (PCR) product. The diagnostic methods of this invention are more particularly described in Section B2 and B3 below.

1. General Aspects of PCR

a. Preparation of Polynucleotide Primers

The term “polynucleotide” as used herein in reference to primers, probes and nucleic acid fragments or segments to be synthesized by primer extension is defined as a molecule comprised of two or more deoxyribonucleotides or ribonucleotides, preferably more than 3. Its exact size will depend on many factors, which in turn depends on the ultimate conditions of use.

The term “primer” as used herein refers to a polynucleotide whether purified from a nucleic acid restriction digest or produced synthetically. The primer is capable of acting as a point of initiation of nucleic acid synthesis when placed under conditions in which synthesis of a primer extension product, complementary to a nucleic acid strand, is induced. Inducing conditions include the presence of nucleotides and an agent for polymerization such as DNA polymerase, reverse transcriptase and the like, and at a suitable temperature and pH. The primer is preferably single stranded for maximum efficiency, but may alternatively be in double stranded form. If double stranded, the primer is first treated to separate it from its complementary strand before being used to prepare extension products. Preferably, the primer is a polydeoxyribonucleotide. The primer must be sufficiently long to prime the synthesis of extension products in the presence of the agents for polymerization. The exact lengths of the primers will depend on many factors, including temperature and the source of primer. For example, depending on the complexity of the target sequence, a polynucleotide primer typically contains 15 to 25 or more nucleotides, although it can contain fewer nucleotides. Short primer molecules generally require cooler temperatures to form sufficiently stable hybrid complexes with template.

The primers used herein are selected to be “substantially” complementary to the different strands of each specific sequence to be synthesized or amplified. This means that the primer must be sufficiently complementary to non-randomly hybridize with its respective template strand. Therefore, the primer sequence may or may not reflect the exact sequence of the template. For example, a non-complementary nucleotide fragment can be attached to the 5′ end of the primer, with the remainder of the primer sequence being substantially complementary to the strand. Such non-complementary fragments typically code for an endonuclease restriction site. Alternatively, non-complementary bases or longer sequences can be interspersed into the primer, provided the primer sequence has sufficient complementarity with the sequence of the strand to be synthesized or amplified to non-randomly hybridize therewith and thereby form an extension product under polynucleotide synthesizing conditions.

Primers of the present invention may also contain a DNA-dependent RNA polymerase promoter sequence or its complement. See for example, Krieg et al, Nucl, Acids Res. 12:7057 (1984); Studier et al, J. Mol. Biol. 189:113 (1986); and Molecular Cloning: A Laboratory Manual. Second Edition, Maniatis et al, eds., Cold Spring Harbor, N.Y. (1989).

When a primer containing a DNA-dependent RNA polymerase promoter is used, the primer is hybridized to the polynucleotide strand to be amplified and the second polynucleotide strand of the DNA-dependent RNA polymerase promoter is completed using an inducing agent such as Escherichia coli DNA polymerase I, or the Klenow fragment of E, coli DNA polymerase. The starting polynucleotide is amplified by alternating between the production of an RNA polynucleotide and DNA polynucleotide.

Primers may also contain a template sequence or replication initiation site for a RNA-directed RNA polymerase. Typical RNA-directed RNA polymerase include the QB replicase described by Lizardi et al, Biotech. 6:1197 (1988). RNA-directed polymerases produce large numbers of RNA strands from a small number of template RNA strands that contain a template sequence or replication initiation site. These polymerases typically give a one million-fold amplification of the template strand as has been described by Kramer et al., J. Mol. Biol. 89:719 (1974).

The polynucleotide primers can be prepared using any suitable method, such as, for example, the phosphotriester or phosphodiester methods see Narang et al., Meth. Enzymol. 68:90, (1979); U.S. Pat. Nos. 4,356,270, 4,458,066, 4,416,988, 4,293,652; and Brown et al, Meth. Enzymol. 68:109, (1979).

The choice of a primer's nucleotide sequence depends on factors such as the distance on the nucleic acid from the hybridization point to the region coding for the mutation to be detected, its hybridization site on the nucleic acid relative to any second primer to be used, and the like.

If the nucleic acid sample is to be enriched for Factor V gene material by PCR amplification, two primers, i.e., a PCR primer pair, must be used for each coding strand of nucleic acid to be amplified. The first primer, having a sequence derived from the coding or sense strand, hybridizes to a nucleotide sequence on the noncoding (anti-sense or minus) strand. With PCR, the first primer thereafter becomes part of the coding (sense or plus) strand. A second primer, having a sequence derived from the noncoding strand, hybridizes to a nucleotide sequence of the coding or sense strand and with PCR, and thereafter it becomes part of the noncoding strand.

In one embodiment, the present invention utilizes a set of polynucleotides that form primers having a priming region located at the 3′-terminus of the primer. The priming region is typically the 3′-most (3′-terminal) 15 to 25 nucleotide bases. The 3′-terminal priming portion of each primer is capable of acting as a primer to catalyze nucleic acid synthesis, i.e., initiate a primer extension reaction off its 3′ terminus. One or both of the primers can additionally contain a 5′-terminal (5′-most) non-priming portion, i.e., a region that does not participate in hybridization to the preferred template.

One or both of the primers can also contain one or more nucleotides in the priming region which is a nonpriming portion, i.e., a region that does not participate in hybridization to the preferred template. Such nucleotides may introduce all or a portion of a restriction endonuclease site that is not present in the preferred template. A preferred primer, used as a second primer, that introduces a portion of a restriction endonuclease site is listed in SEQ ID NO 24 introducing a Hind III restriction site in a mutated Factor V allele of this invention.

In PCR, each primer works in combination with a second primer to amplify a target nucleic acid sequence. The choice of PCR primer pairs for use in PCR is governed by considerations as discussed herein for producing Factor V gene regions. Useful priming sequences for amplifying both Factor V genomic DNA and cDNA from a patient sample are described below in Section B2 and in Example 1.

b. Polymerase Chain Reaction

Factor V genes are comprised of polynucleotide coding strands, such as mRNA and/or the sense strand of genomic DNA. If the genetic material to be assayed is in the form of double stranded genomic DNA, it is usually first denatured, typically by melting, into single strands. The nucleic acid is subjected to a PCR amplification by treating (contacting) the sample with a PCR primer pair, each member of the pair having a preselected nucleotide sequence based on the design requirements as described in Section Bla above. Primers comprising a primer pair are capable of initiating a primer extension reaction by hybridizing to a template nucleotide sequence, preferably at least about 10 nucleotides in length, more preferably at least about 15 nucleotides in length and most preferably 20 nucleotides in length, that are present and preferably conserved within a Factor V allele template.

The first primer of a PCR primer pair is sometimes referred to herein as the “sense primer” because it is derived from the sense or coding strand and it hybridizes to the anti-sense (noncoding or minus) strand of a nucleic acid, i.e., a strand complementary to a coding strand. Accordingly, the second primer of a PCR primer pair is sometimes referred to herein as the “anti-sense primer” because it is derived from the anti-sense strand and it hybridizes to a sense (coding or plus) strand of a nucleic acid. With PCR, the anti-sense primer becomes a part of the amplified anti-sense strand.

The PCR reaction is performed by mixing the PCR primer pair, preferably a predetermined amount thereof, with the nucleic acids of the sample, preferably a predetermined amount thereof, in a PCR buffer to form a PCR reaction admixture. The admixture is thermocycled for a number of cycles, which is typically predetermined, sufficient for the formation of a PCR amplification product, thereby enriching the sample to be assayed for Factor V genetic material. Thus, as defined herein an amplification product of this invention results from the amplification of a Factor V nucleic acid, either genomic DNA or cDNA, with a particular primer pair.

PCR is typically carried out by thermocycling i.e., repeatedly increasing and decreasing the temperature of a PCR reaction admixture within a temperature range whose lower limit is about 30° C. to about 70° C. and whose upper limit is about 90° C. to about 100° C. The increasing and decreasing can be continuous, but is preferably phasic with time periods of relative temperature stability at each of temperatures favoring polynucleotide synthesis, denaturation and hybridization.

A plurality of first primers and/or a plurality of second primers can be used in each amplification, e.g., one species of a first primer can be paired with a number of different second primers to form several different primer pairs.

Alternatively, an individual pair of first and second primers can be used. For example, in amplifying Factor V genomic DNA as described in Example 1B and 1C, the first primer having a nucleotide sequence shown in SEQ ID NO 4 is separately paired with either of the second primers having the respective sequences shown in SEQ ID NOs 5 and 24. The determination of which pairing is to be utilized for a particular amplification depends on the assay method selected for screening for the presence or absence of the mutation, i.e., whether the assay method is based on restriction digestion of amplified products by a Mnl I or a Hind III restriction endonuclease as discussed below in Section B3.

In any case, the amplification products of amplifications using the same or different combinations of first and second primers can be combined for assaying for the Factor V guanine to adenine point mutation of this invention.

The PCR reaction is performed using any suitable method. Generally it occurs in a buffered aqueous solution, i.e., a PCR buffer, preferably at a pH of 7-9, most preferably about 8. Preferably, a molar excess (for genomic nucleic acid, usually about 106:1 primer:template) of the primer is admixed to the buffer containing the template strand. A large molar excess is preferred to improve the efficiency of the process.

The PCR buffer also contains the deoxyribonucleotide triphosphates (polynucleotide synthesis substrates) DATP, dCTP, dGTP, and dTTP and a polymerase, typically thermostable, all in adequate amounts for primer extension (polynucleotide synthesis) reaction. The resulting solution (PCR admixture) is heated to about 90° C.-100° C. for about 1 to 10 minutes, preferably from 1 to 5 minutes. After this heating period, the solution is allowed to cool to 56° C., which is preferable for primer hybridization. A more preferred primer hybridization temperature is 60° C. Other aspects of hybridization conditions and requirements are described in Section B3c.

The synthesis reaction may occur at from room temperature up to a temperature above which the polymerase (inducing agent) no longer functions efficiently. Thus, for example, if E, coli DNA polymerase I is used as inducing agent, the temperature is generally no greater than about 40° C. The thermocycling is repeated until the desired amount of PCR product is produced. An exemplary PCR buffer comprises the following: 50 mM KCl; 10 mM Tris-HCl; pH 8.3; 1.5 mM MgCl₂; 0.001% (wt/vol) gelatin, 200 μM DATP; 200 μM dTTP; 200 μM dCTP; 200 μM dGTP; and 2.5 units Thermus aguaticus DNA polymerase (U.S. Pat. No. 4,889,818) per 100 microliters of buffer.

The inducing agent may be any compound or system which will function to accomplish the synthesis of primer extension products, including enzymes. Suitable enzymes for this purpose include, for example, E, coli DNA polymerase I, Klenow fragment of E, coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Examples of heat-stable enzymes include Thermus aguaticus DNA polymerase, Pyrococcus furiosus DNA polymerase, and Thermatoga maratima DNA polymerase, among others. Generally, the synthesis will be initiated at the 3′ end of each primer and proceed in the 5′ direction along the template strand, until synthesis terminates, producing molecules of different lengths or of the same length. There may be inducing agents, however, which initiate synthesis at the 5′ end and proceed in the above direction, using the same process as described above.

The inducing agent also may be a compound or system which will function to accomplish the synthesis of RNA primer extension products, including enzymes. In preferred embodiments, the inducing agent may be a DNA-dependent RNA polymerase such as T7 RNA polymerase, T3 RNA polymerase or SP6 RNA polymerase. These polymerases produce a complementary RNA polynucleotide. The high turn over rate of the RNA polymerase amplifies the starting polynucleotide as has been described by Chamberlin et al, The Enzymes, ed. P. Boyer, PP. 87-108, Academic Press, New York (1982). Amplification systems based on transcription have been described by Gingeras et al, in PCR Protocols. A Guide to Methods and Applications, pp. 245-252, Academic Press, Inc., San Diego, Calif. (1990).

If the inducing agent is a DNA-dependent RNA polymerase and therefore incorporates ribonucleotide triphosphates, sufficient amounts of ATP, CTP, GTP and UTP are admixed to the primer extension reaction admixture and the resulting solution is treated as described above.

The newly synthesized strand and its complementary nucleic acid strand form a double-stranded molecule which can be used in the succeeding steps of the process.

PCR amplification methods are described in detail in U.S. Pat. Nos. 4,683,192, 4,683,202, 4,800,159, and 4,965,188, and at least in several texts including “PCR Technology: Principles and Applications for DNA Amplification”, H. Erlich, ed., Stockton Press, New York (1989); and “PCR Protocols: A Guide to Methods and Applications”, Innis et al, eds., Academic Press, San Diego, Calif. (1990). Exemplary PCR methods for use in this invention are described in Example 1.

In an alternative embodiment, two pairs of first and second primers are used per amplification reaction. The amplification reaction products obtained from a plurality of different amplifications, each using a plurality of different primer pairs, can then be combined or assayed separately.

2. PCR Amplification of Factor V Nucleic Acid preparations

a. Genomic DNA

In view of the foregoing, the present invention contemplates a screening method comprising treating, under amplification conditions, a sample of genomic DNA isolated from a human with a PCR primer pair for amplifying a region of human genomic DNA containing nucleotide position 205 in exon 10 of the Factor V gene. The genomic DNA sample is obtained from cells, typically peripheral blood leukocytes. Amplification conditions include, in an amount effective for polypeptide synthesis, the presence of PCR buffer and a thermocycling temperature.

The PCR amplification product thus produced is then assayed as described below in Section B3 for the presence of a point mutation characterized as a change from a guanine nucleotide to an adenine nucleotide at nucleotide position 205 in exon 10 of the Factor V gene.

In one preferred embodiment, a PCR primer pair produces an amplification product containing a restriction endonuclease site if the normal allele is present. However, if a mutant allele containing the guanine to adenine point mutation is amplified, the same PCR primer pair produces an amplification product that does not contain a restriction endonuclease site.

Preferably, the above-described PCR primer pair comprises a first primer that hybridizes to noncoding strand of exon 10 at a location 3′ to nucleotide position 205 in exon 10 in the noncoding strand (a position equivalent to 5′ of the mutation on the complementary coding strand), and a second primer that hybridizes to a coding strand of intron 10 at a location 3′ to nucleotide position 205 in exon 10 of the coding strand. A preferred first primer, FV7, is represented by the sequence, 5′-CATACTACAGTGACGTGGAC-3′ (SEQ ID NO 4), and a preferred second primer, FVINT102, is represented by the sequence, 5′-TGTTCTCTTGAAGGAAATGC-3′ (SEQ ID NO 5).

In one embodiment of the invention, the PCR amplification product resulting from the above-described PCR primer pair contains a continuous nucleotide sequence written from 5′ to 3′ direction represented by the sequence, 5′-GACAGGCNAGG-3′ (SEQ ID NO 1) wherein N is either G, as in a normal Factor V gene, or A, as in a mutant gene, or a fragment thereof containing nucleotide position 205 in exon 10 of Factor V genomic DNA. Exemplary nucleotide sequences of Factor V genomic DNA containing the preferred nucleotide sequence of SEQ ID NO 1 are given in FIGS. 2A (SEQ ID NO 2) and 2B (SEQ ID NO 3), that are respectively normal and mutated PCR amplified genomic DNA fragments generated with the PCR primer pair FV7 and FVINT102.

If the normal allele is amplified, the resulting amplification product contains the nucleotide guanine at nucleotide position 205 in exon 10 of the Factor V gene. In contrast, amplification of the mutant allele produces an amplification product containing the nucleotide adenine at nucleotide position 205 in exon 10 of the Factor V gene.

If both the normal and mutant alleles are present, as in the heterozygous state, the PCR primer pair produces an amplification product containing a guanine at nucleotide position 205 in exon 10 of the Factor V gene in the normal allele and an adenine at nucleotide position 205 in exon 10 of the Factor V gene in the mutant allele.

With the primer pairs of this invention, a preferred amplified nucleotide region of the Factor V gene of a normal allele lacking the point mutation contains the nucleotide sequence corresponding to 5′-ACAGGCGAGG-3′ (SEQ ID NO 6), or a fragment thereof. The term region refers to the nucleotides in the amplification product actually amplified by using a preselected primer pair. The phrase “a fragment thereof” means that a region of amplified nucleotides forming an amplification product contains a portion of the noted sequences. The presence of the guanine nucleotide corresponding to nucleotide position 205 in exon 10 confers a Mnl I restriction endonuclease digestion site present in a normal allele.

The corresponding preferred amplified nucleotide region of the Factor V gene of an affected allele contains the new point mutation as underlined within the nucleotide sequence corresponding to 5′-ACAGGCAAGG-3′ (SEQ ID NO 7), or a fragment thereof. The presence of the adenine nucleotide corresponding to nucleotide position 205 in exon 10 destroys a Mnl I restriction endonuclease digestion site present in a normal allele.

Therefore, the presence of the point mutation destroys the ability of the restriction endonuclease Mnl I to digest the Factor V DNA in the amplified nucleotide region. As a result, the differential Mnl I restriction digestion patterns between a normal allele and an affected allele allows for the determination of the presence of the point mutation in a Factor V gene allele in a patient having APC resistance.

Thus, both restriction digestion and nucleotide sequence analysis, as described in Section B3, of amplification products containing nucleotide position 205 in exon 10 of the Factor V gene provides for the determination between a homozygous normal, a homozygous mutant or heterozygous state. Such analyses are described in Examples 1A-1B.

In another aspect of screening genomic DNA from a patient, an alternative PCR primer pair to that described above is utilized to produce an amplification product containing a restriction endonuclease site if the point mutation is present. In other words, a restriction endonuclease site is created in an amplification product that is dependent on the presence of the guanine to adenine point mutation in nucleotide position 205 of exon 10. Without the presence of the adenine nucleotide, as occurs in the normal PCR amplified allele having the normal guanine nucleotide at that location, the restriction site is lost resulting in the inability to create a restriction digestion product at that site.

Therefore, this alternative embodiment provides a different restriction pattern than that obtained with the PCR primer pair, FV7 and FVINT102, used for a Mnl I restriction analysis. Analysis of a patient's genomic DNA with both approaches allows for the confirmation of a genetic diagnosis by two independent but complementary means.

A preferred first primer designated FV7 and second primer designated FV506tst2 of a primer pair for amplifying normal and affected alleles, to generate a restriction site in the latter, have the respective nucleotide sequences 5′-CATACTACAGTGACGTGGAC-3′ (SEQ ID NO 4) and 5′-TTACTTCAAGGACAAAATACCTGTAAAGCT-3′ (SEQ ID NO 24).

The second primer was designed to create a restriction endonuclease site utilizing nucleotide position 205 of exon 10 having the adenine point mutation. In order to create a preselected restriction endonuclease site that utilizes the adenine point mutation, the second primer FV506tst2 was further designed to introduce three additional point mutations into the nucleotide sequences amplified from the provided template genomic DNA. These additional mutations are generated in the amplified products corresponding to nucleotide position 208 extending to nucleotide position 210 (5′-CTT-3′ of the coding or sense strand is changed to 5′-GAA-3′).

As a result of amplification with the above-described primer pair, a restriction endonuclease site, specifically a Hind III site, is created utilizing nucleotide position 205 of exon 10 where it did not naturally exist in either the normal or mutant alleles. The only naturally occurring restriction site in exon 10 utilizing the guanine nucleotide at nucleotide position 205 is the previously discussed Mnl I restriction site in the normal allele which is dependent on the presence of a guanine nucleotide at nucleotide position 205.

With amplification of genomic DNA with primer pairs FV7 and FV506tst2, a resultant preferred amplified nucleotide region of the Factor V gene of an affected allele having the point mutation contains nucleotide sequence 5′-AAGCTT-3′ (SEQ ID NO 23), or a fragment thereof, the sequence of which is the Hind III restriction endonuclease site. The corresponding preferred amplified nucleotide region of the Factor V gene of a normal allele that lacks the point mutation contains the nucleotide sequence 5′-GAGCTT-3′ (SEQ ID NO 25), or a fragment thereof.

An amplified mutant allele nucleotide region having SEQ ID NO 23 and consisting essentially of the sequence shown in SEQ ID NO 19 therefore contains a Hind III restriction endonuclease digestion site. The corresponding amplified nucleotide region from a normal allele having a guanine nucleotide, consisting essentially of the sequence shown in SEQ ID NO 18, does not contain the requisite Hind III site. Therefore, differential Hind III restriction digestion patterns of genomic DNA amplified with the primer pairs of SEQ ID NOs 4 and 24 provide an alternative method for the ability to determine the presence or absence of the point mutation from genomic DNA of a patient. Such an analysis is described in Example 1C.

Also contemplated for use in practicing the methods of this invention are other first and second primers that can be designed to produce an amplification product of this invention. Such primers can be of the category containing the Mnl I primer pair, as previously described, that amplify normal and mutant genomic alleles that utilize restriction endonuclease sites in normal and not mutant alleles. Moreover, these primers are designed to rely on the naturally occurring nucleotide sequence of the template DNA, either normal or mutant, without introducing additional mutations. These types primers are designed by selecting regions of the nucleotide sequence including or around the guanine to adenine point mutation site of this invention that provide an amplification product for subsequent analysis by one of the methods as described in Section B3 below.

However, the design of primers of a primer pair to amplify a region of Factor V nucleic acid is not limited to having a restriction site localized at the position of the point mutation of this invention. As long as the normal and mutant amplified products can be digested to form differential restriction digestion patterns, any primer pair can be utilized to form amplification products.

Other primers can also be similarly designed to the category of primers containing the Hind III noncoding primer, FV506tst2, as previously described that utilize the guanine to adenine point mutation to create a restriction endonuclease site not normally present at that site. Similarly, primers that introduce additional mutations into the amplified products are contemplated irrespective of whether the guanine or adenine nucleotide is present at nucleotide position 205 of exon 10.

Moreover, as contemplated for use in this invention, the design of the primers is not limited to those for which a restriction endonuclease digestion assay method is required to determine the presence or absence of the guanine to adenine point mutation. As such, primers can be designed to amplify a region of Factor V genetic material for nucleotide sequence analysis alone, as described in Example 1A, for restriction digestion analysis, as described in Examples 1B and 1C, or for analysis by nucleic acid hybridization methods, as described below in Section B3.

b. cDNA

In another preferred embodiment, the invention contemplates a screening method comprising treating, under amplification conditions, a sample of cDNA, synthesized from messenger RNA (mRNA) isolated from a patient, with a PCR primer pair for amplifying a region of human cDNA containing nucleotide position 1691 of Factor V cDNA.

Where mRNA is used, the cells are lysed under RNase inhibiting conditions. In one embodiment, the first step is to isolate the total cellular mRNA. Poly A+ mRNA can then be selected by hybridization to oligo-dT cellulose.

A complementary strand of DNA, referred to as cDNA, is thereafter synthesized with methods well known to one of ordinary skill in the art and as described in Example 1D. Since the synthesized cDNA strand is generated from mRNA, it is the noncoding or anti-sense strand.

Amplification conditions for amplifying the resultant cDNA include, in an amount effective for polypeptide synthesis, the presence of PCR buffer and a thermocycling temperature.

Preferably, the PCR primer pair for amplifying cDNA comprises a first primer that hybridizes to a noncoding strand of the cDNA at a location 3′ to nucleotide 1691 of the noncoding cDNA strand, and a second primer that hybridizes to a coding strand of the cDNA at a location 3′ to nucleotide position 1691 of the coding cDNA strand.

In a preferred embodiment, the PCR primer pair produces an amplification product containing a restriction endonuclease site if amplifying normal cDNA. However, in mutant cDNA, the PCR primer pair produces an amplification product which does not contain a restriction endonuclease site.

However, as discussed above for genomic DNA amplification, other primer pairs are contemplated for use in preparing amplified cDNA to detect a Factor V mutation of this invention irrespective of the presence of a restriction endonuclease site.

A preferred primer pair for amplifying Factor V cDNA to produce a PCR amplification product comprises a first primer, FV13, is represented by the sequence, 5′-CAGGAAAGGAAGCATGTTCC-3′ (SEQ ID NO 10), and a preferred second primer, FV2, is represented by the sequence, 5′-TGCCATTCTCCAGAGCTAGG-3′ (SEQ ID NO 11).

The PCR amplification product of 2297 base pairs in size thus produced from the FV13/FV2 primer pair is then assayed preferably by nucleotide sequence analysis as described below in Section B3b for the presence of a point mutation characterized as a change from a guanine nucleotide to an adenine nucleotide at nucleotide position 1691 of Factor V cDNA.

Another preferred primer pair for amplifying Factor V cDNA to produce a PCR amplification product comprises a first primer, FV7, that is represented by the sequence, 5′-CATACTACAGTGACGTGGAC-3′ (SEQ ID NO 4) and a second primer. FV8A, that is represented by the nucleotide sequence, 5′-TGCTGTTCGATGTCTGCTGC-3′ (SEQ ID NO 12). The resultant amplification product is 124 base pairs in size.

In preferred embodiments, the FV7 and FV8A primer pairs are used in PCR amplifications with the cDNA amplification products from the PCR with primer pair FV13 and FV2 used as the template. This procedure is also referred to as a two step or sequential PCR as the amplification products of the first reaction are then used as templates for the second reaction where a separate PCR primer pair is utilized for the latter reaction.

The two-step PCR procedure provides the advantages of reducing potential spurious and nonspecific priming by PCR primers to regions of template not contemplated as specific priming sites. In other words, generating a preferred small PCR amplification product in a one-step PCR amplification when the template is large, such as the approximately 6900 base pair Factor V cDNA nucleotide sequence, may result in a heterogeneous mixture of amplification products that include the desired products as well as those from regions of the template cDNA that are not expected.

Therefore, to increase the specificity of priming reactions and increase the yield of preferred amplification products, the two-step or sequential PCR method is preferred particularly when the template for amplification is large.

In alternative embodiments, however, producing cDNA amplification products with the cDNA specific primer pair, preferably the primer pair FV7 and FV8A, is also performed with the use of the intact Factor V cDNA template synthesized from messenger RNA (mRNA).

The PCR amplification product thus produced from the FV7/FV8A primer pair is then assayed preferably by nucleotide sequence analysis as described below in Section B3b and more preferably by Mnl I restriction digestion analysis as described in Section B3a for the presence of a point mutation characterized as a change from a guanine nucleotide to an adenine nucleotide at nucleotide position 1691 of Factor V cDNA.

Preferably, the PCR product contains a continuous nucleotide sequence written from 5′ to 3′ direction represented by the sequence, 5′-GACAGGCNAGG-3′ (SEQ ID NO 1) wherein N is either a guanine (G), as in a normal Factor V gene, or an adenine (A), as in a mutant gene, or a fragment thereof containing nucleotide 1691 of Factor V cDNA.

Thus, a PCR primer pair produces an amplification product containing the nucleotide guanine at nucleotide 1691 of normal Factor V cDNA whereas in mutant cDNA nucleotide, an adenine nucleotide is substituted for guanine.

With the preferred primer pairs described herein for amplifying cDNA, a preferred amplified nucleotide region of the Factor V gene of a normal allele lacking the point mutation contains the nucleotide sequence corresponding to 5′-ACAGGCGAGG-3′ (SEQ ID NO 6), or a fragment thereof. As with genomic DNA, the presence of the guanine nucleotide corresponding to nucleotide position 1691 of the cDNA, confers a Mnl I restriction endonuclease digestion site present in a normal allele.

The corresponding preferred amplified mutant nucleotide region of cDNA contains the nucleotide sequence corresponding to 5′-ACAGGCAAGG-3′ (SEQ ID NO 7), or a fragment thereof. The presence of the adenine nucleotide corresponding to nucleotide position 1691 of the cDNA destroys a Mnl I restriction endonuclease digestion site present in normal cDNA.

In normal cDNA, the amplified nucleotide region resulting from amplification with primer pairs FV13 and FV2 described above consists essentially of a 2297 base pair nucleotide sequence shown in SEQ ID NO 27 having a guanine nucleotide at cDNA nucleotide position 1614, which corresponds to nucleotide position 1691 in intact Factor V cDNA. The basis for the discrepancy in the nucleotide position of the guanine nucleotide being at 1614 in SEQ ID NO 27 as compared to 1691 in intact Factor V cDNA stems from the convention for numbering the amplified cDNA as shown in SEQ ID NO 27. In the latter, nucleotide position 1 corresponds to nucleotide position 78 in Factor V cDNA as shown in SEQ ID NO 13 and in the both FIGS. 6A and 6B.

In cDNA containing the point mutation, the corresponding amplified nucleotide region consists essentially of a 2297 base pair nucleotide sequence shown in SEQ ID NO 28 having an adenine nucleotide at nucleotide position 1614 that corresponds to nucleotide position 1691 in intact Factor V cDNA as described above. The presence of the latter destroys a normal Mnl I restriction endonuclease site.

Both the normal and mutant amplified cDNA nucleotide sequences are shown in one sequence in SEQ ID NO 17 where the nucleotide at position 1614 (corresponding to nucleotide position 1691 of intact cDNA as previously discussed) is indicated as an “n” wherein “n” is either a guanine nucleotide in the normal Factor V cDNA or an adenine nucleotide indicating the point mutation in the mutant Factor V cDNA.

In normal cDNA, the amplified nucleotide region resulting from amplification with primer pairs FV7 and FV8A described above consists essentially of a 124 base pair nucleotide sequence shown in SEQ ID NO 13, from nucleotide position 1601 to 1724 having a guanine nucleotide at cDNA nucleotide position 1691. In cDNA containing the point mutation, the corresponding amplified nucleotide region consists essentially of a nucleotide sequence shown in SEQ ID NO 26, from nucleotide position 1601 to 1724 having an adenine nucleotide at the 1691 nucleotide position. The presence of the latter destroys a normal Mnl I restriction endonuclease site.

Thus, in the method for identifying a Factor V genetic mutation in Factor V cDNA, since the Mnl I site is inherently present in normal Factor V nucleic acid, the PCR primer pair produces a cDNA amplification product containing a restriction endonuclease site if the point mutation is not present.

In one embodiment, the resultant amplification products are then treated, under restriction conditions, with a restriction endonuclease, preferably Mnl I, that recognizes the restriction site and cleaves the cDNA amplification product if the point mutation is absent. As a result, the restriction digestion patterns between a normal allele and an affected allele allows for the determination of the presence or absence of the point mutation in cDNA generated from a mRNA sample.

In another embodiment, the same amplified products are assayed by nucleotide sequence analysis or nucleic acid hybridization techniques to determine the presence or absence of the point mutation.

Also contemplated as an amplification product of this invention produced by a primer pair designed to allow such a product to be amplified is the full length Factor V cDNA having the guanine to adenine point mutation at nucleotide position 1691. The nucleotide sequence of this amplified cDNA product is shown in SEQ ID NO 26, from nucleotide position 9 to nucleotide position 6917. The 5′ and 3′ terminal 8-mer Eco RI cloning linkers as previously discussed and shown in the sequence do not comprise the Factor V cDNA.

Thus, both restriction analysis and nucleotide sequence as respectively described in Sections B3a and B3b provide for the determination of the presence or absence of the guanine to adenine point mutation of this invention in a cDNA amplification products. Such nucleotide sequencing and restriction digestion analyses are respectively described in Examples 1D and 1E.

3. Methods for Assaying Factor V Nucleic Acid Preparations

As contemplated for use in assaying an amplification product produced by the methods of this invention, various assay methods as described below provide a means to screen a patient for a Factor V gene mutation associated with APC resistance. In particular, the assay methods allow for the determination of the presence or absence of the Factor V guanine to adenine point mutation at nucleotide position 205 in exon 10 and at nucleotide position 1691 in the corresponding cDNA.

a. Restriction Endonuclease Digestion Analysis

In a preferred embodiment, assaying comprises treating, under restriction conditions, an amplification product with a restriction enzyme that recognizes a restriction site in the product and cleaves the amplification product at a specific site to form restriction products. The resultant restriction digestion products then detected as described below.

In the homozygous state, either the normal or mutant allele is present and is detected by the method as described herein. In the heterozygous state, both the normal and mutant allele is present and detected by the method as described herein.

As contemplated for use in the methods of this invention, restriction digestion of an amplified product is performed under optimal restriction conditions that are dictated by the type and specificity of the restriction endonuclease that is used. In general, the restriction conditions, that include the amount of restriction endonuclease, restriction buffer, digestion temperature and digestion time and the like, that are optimal for a particular restriction endonuclease are provided in the product material with manufacturer's instructions. Different manufacturers of restriction endonucleases recommend significantly different digestion conditions, even for the same endonuclease. As a result, since most manufacturers have optimized the reaction conditions for their particular products, for use in this invention, the restriction conditions employed are governed by the selection of restriction endonuclease and the manufacturers' instructions. Exemplary restriction conditions for Mnl I and Hind III restriction endonucleases are respectively provided in Examples 1B and 1C.

In the context of assaying by restriction endonuclease digestion analysis, the invention further require a means for detecting the formed restriction digestion products. Thus, in a preferred embodiment, the presence of restriction products can be detected by electrophoresis through agarose or polyacrylamide gels which is a standard method used to separate and identify DNA fragments (Sambrook et al, Molecular Cloning. A Laboratory Manual, 2nd ed., Cold Spring Harbor Press, Cold Spring Harbor, N.Y., 1989). The technique is rapid and simple to perform. DNA fragments applied to the agarose or polyacrylamide gel are separated electrophoretically based upon their molecular weight.

The position of the DNA fragment within the gel subsequent to electrophoresis can be directly determined by staining of the DNA with a fluorescent intercalating dye such as ethidium bromide and examination of the gel under ultraviolet light (Sharp et al, Biochem. 12:3055, 1973). Thus, the molecular weights of DNA fragments subsequent to incubation with Mnl I can be determined by comparison of the migration of the resultant DNA fragments to commercially available DNA molecular weight standards.

Preferably, the restriction endonuclease used to assay the presence or absence of a guanine to adenine point mutation of this invention in an amplified product is the type I restriction endonuclease Mnl I and the restriction site is represented by the sequence, 5′-ACAGGCGAGG-3′ (SEQ ID NO 6; Brinkley et al, Gene 100:267, 1991) wherein the guanine nucleotide which is underlined corresponds to nucleotide position 205 in exon 10 of Factor V genomic DNA and nucleotide position 1691 of Factor V cDNA. In the mutant allele, the nucleotide sequence at position 205 in exon 10 of the Factor V gene is represented by the sequence comprising 5′-ACAGGCAAGG-3, (SEQ ID NO 7), where the underlined nucleotide indicates the adenine point mutation.

Mnl I recognizes a specific double-stranded nucleotide sequence (restriction endonuclease recognition site or recognition site) and cleaves both strands of the double-stranded DNA at a position within the nucleotide sequence that is not contained within the recognition site. Cleavage of the double-stranded nucleotide sequence by a restriction endonuclease results in restriction products.

The nucleotide sequences representing the recognition sites of type I restriction endonucleases are not palindromic.

By convention, the recognition site for Mnl I is represented by the double-stranded nucleotide sequence as follows:

5′-CCTCNNNNNNN-3′ (SEQ ID NO 20)

3′-GGAGNNNNNN-5′ (SEQ ID NO 21)

wherein N is any nucleotide (Brinkley et al, Gene 100:267, 1991).

Thus, the coding nucleotide sequence 5′-ACAGGCGAGG-3′ (SEQ ID NO 6) in exon 10, the latter shown in SEQ ID NO 15) of the Factor V gene from nucleotide positions 199 to 208 and in the cDNA of Factor V cDNA from nucleotide positions 1685 to 1694 corresponds to the Mnl I sequence shown in SEQ ID NO 21 as read from 5′ to 3′. As such, the sequence is a single-strand of the double-stranded DNA representing a Mnl I recognition site.

Since the cleavage pattern by Mnl I results in a 3′ one base pair overhang as shown above, the convention therefore adopted for determining the fragment sizes generated by Mnl I digestion in this invention is based upon the Mnl I restriction digestion cleavage of the coding or sense strand of the amplification products which may have either of the above nucleotide sequence specificities. For example, as discussed in Example 1B, with either amplified genomic or cDNA from Factor V, the 5′ Mnl I site has the 3′ one-base overhang in the coding strand while the 3′ Mnl I site that contains nucleotide 205 in exon 10 (nucleotide 1691 in cDNA) is just the reverse having the 3′ one-base overhang in the noncoding or anti-sense strand. The coding strand of the 3′ Mnl I site therefore is the shorter cleavage product. Calculation of the restriction products resulting from Mnl I digestion based on the coding strand cleavage sites is either one base plus or minus depending on the double-stranded Mnl I site.

An allele which contains the Mnl I recognition site at nucleotide positions 199 to 208 in exon 10 of the Factor V gene is a normal allele. Amplification products derived from the Factor V gene of a normal allele are cleaved in the presence of Mnl I to form restriction products.

A change in the nucleotide sequence representing a Mnl I recognition site from a guanine to an adenine in exon 10 of the Factor V gene at nucleotide position 205 and in the Factor V cDNA at nucleotide position 1691, therefore, eliminates the Mnl I recognition site. As a result, an allele which does not contain a Mnl I recognition site at nucleotide positions 199 to 208 in exon 10 of the Factor V gene is a mutant allele. Amplification products derived from the Factor V gene of a mutant allele which do not contain a Mnl I recognition site at nucleotide positions 199 to 208 in exon 10 of the Factor V gene are therefore not cleaved in the presence of Mnl I to form restriction products.

Therefore, in the homozygous mutant Factor V genotype, where both alleles are mutant, the restriction endonuclease Mnl I does not cleave at position 205 in exon 10 of the Factor V gene. Therefore, a homozygous mutant genotype can be detected by the lack of a Mnl I restriction endonuclease site at and including nucleotide position 205 in amplification products.

In the heterozygous Factor V genotype, the presence of both the normal and mutant alleles are detected by the respective presence of both cleaved and uncleaved DNA with incubation of amplification products derived from the Factor V gene of the normal and mutant alleles in the presence of the restriction endonuclease Mnl I.

Homozygous normal Factor V alleles, in contrast, are both digested at the Mnl I site that includes nucleotide position 205 in exon 10.

Thus, the differential Mnl I digestion patterns observable upon electrophoresis allows for the determination of the genotype of a patient's sample with respect to the guanine to adenine point mutation of this invention.

Exemplary Mnl I restriction digestion analysis of both genomic DNA and cDNA PCR amplification products is respectively described in Examples 1B and 1E.

In an alternative embodiment, the presence of the guanine to adenine point mutation is detected by digestion or lack of digestion of an amplification product with Hind III.

Hind III is a type II restriction endonuclease that recognizes a specific double-stranded nucleotide sequence (restriction endonuclease recognition site or recognition site) and cleaves both strands of the double-stranded DNA at a position within the recognition site.

Hind III recognizes double-stranded DNA represented by the following sequence:

5′-AAGCTT-3′ (SEQ ID NO 23).

The specific cleavage of type II restriction endonucleases generates restriction products containing a specific number of nucleotides. Thus, Hind III restriction products containing a specific number of nucleotides are generated by cleavage of double-stranded DNA with Hind III.

The Hind III nucleotide sequence 5′-AAGCTT-3′ (SEQ ID NO 23) in an mutant allelic amplified product corresponds to nucleotide positions 205 to 210 in exon 10. Incubation of double-stranded DNA containing the sequence given in SEQ ID NO 23 in the presence of the restriction endonuclease Hind III results in the cleavage of both strands of the DNA to generate the following structure:

5′-A-3′

3′-TTCGA-5′ (SEQ ID NO 23, from nucleotide position 2 to 6 as shown in the 5′ to 3′ direction).

The amplified normal allelic counterpart contains the nucleotide sequence 5′-GAGCTT-3′ (SEQ ID NO 25). This sequence does not represent a Hind III recognition site and therefore is not cleaved at nucleotide position 205 when incubated in the presence of the restriction endonuclease Hind III.

Thus, the differential Hind III digestion patterns observable upon electrophoresis allow for the determination of the genotype of a patient's sample with respect to the guanine to adenine point mutation of this invention.

Exemplary Hind III restriction digestion analysis of genomic DNA PCR amplification products is described in Example 1C.

Other restriction endonuclease assay methods are contemplated for use in this invention as required by the design of alternative primers utilizing new restriction site specificities.

b. Sequence Analysis of Factor V Nucleic Acid Preparations

In another embodiment of this invention, assaying comprises determining the nucleotide sequence of the amplification product at nucleotide position 205 in exon 10 of the Factor V gene and at nucleotide position 1691 of the Factor V cDNA as described in Examples 1A and 1D, respectively. Nucleic acid sequence analysis is an alternative approach to restriction digestion assay methods to assay a Factor V nucleic acid preparation, including an amplified product resulting from PCR as described in the methods of this invention. A nucleic acid sequence analysis determination is also contemplated for non-PCR amplified nucleic acid samples isolated from patients.

For either of the above embodiments, such an analysis on a selected nucleic acid sample is approached by a combination of (a) physiochemical techniques, based on the hybridization or denaturation of a probe strand plus its complementary target, and (b) enzymatic reactions with endonucleases, ligases, and polymerases. Nucleic acid can be assayed as either DNA or RNA. With DNA, the genetic potential of individual humans is analyzed and with RNA, the expressed information of particular cells is determined.

For sequencing, a sequence in the template DNA may be known, such as where the primer to be formed can hybridize to known Factor V sequences and initiates primer extension into a region of DNA for sequencing purposes, or where previous sequencing has determined a region of nucleotide sequence and the primer is designed to extend from the recently sequenced region into a region of unknown sequence. This latter process has been referred to a “directed sequencing” because each round of sequencing is directed by a primer designed based on the previously determined sequence.

Exemplary sequencing approaches for use in analyzing Factor V genetic material from a patient sample to determine the presence or absence of the Factor V guanine to adenine point mutation of this invention are described in Example 1. A preferred sequencing primer is designated FV23, the sequence of which is listed in SEQ ID NO 22.

c. Detection of Factor V Nucleic Acid Preparations for Hybridization Assays

In assays using nucleic acid hybridization, detecting the presence of a DNA duplex in a process of the present invention can be accomplished by a variety of means.

In one approach for detecting the presence of a DNA duplex, an oligonucleotide that is hybridized in the DNA duplex includes a label or indicating group that will render the duplex detectable. Typically such labels include radioactive atoms, chemically modified nucleotide bases, and the like.

The oligonucleotide can be labeled, i.e., operatively linked to an indicating means or group, and used to detect the presence of a specific nucleotide sequence in a target template.

Radioactive elements operatively linked to or present as part of an oligonucleotide probe (labeled oligonucleotide) provide a useful means to facilitate the detection of a DNA duplex. A typical radioactive element is one that produces beta ray emissions. Elements that emit beta rays, such as ³H, ¹²C, ³²p and ³⁵S represent a class of beta ray emission-producing radioactive element labels. A radioactive polynucleotide probe is typically prepared by enzymatic incorporation of radioactively labeled nucleotides into a nucleic acid using DNA kinase.

Alternatives to radioactively labeled oligonucleotides are oligonucleotides that are chemically modified to contain metal complexing agents, biotin-containing groups, fluorescent compounds, and the like.

One useful metal complexing agent is a lanthanide chelate formed by a lanthanide and an aromatic betadiketone, the lanthanide being bound to the nucleic acid or oligonucleotide via a chelate forming compound such as an EDTA-analogue so that a fluorescent lanthanide complex is formed. See U.S. Pat. Nos. 4,374,120, 4,569,790 and published Patent Application Nos. EP0139675 and WO87/02708.

Biotin or acridine ester-labeled oligonucleotides and their use to label polynucleotides have been described. See U.S. Pat. No. 4,707,404, published Patent Application EP0212951 and European Patent No. 0087636. Useful fluorescent marker compounds include fluorescein, rhodamine, Texas Red, NBD and the like.

A labeled oligonucleotide present in a DNA duplex renders the duplex itself labeled and therefore distinguishable over other nucleic acids present in a sample to be assayed. Detecting the presence of the label in the duplex and thereby the presence of the duplex, typically involves separating the DNA duplex from any labeled oligonucleotide probe that is not hybridized to a DNA duplex. Preferred oligonucleotides for use in forming a DNA duplex are nucleotide sequences corresponding to SEQ ID NOs 6 and 7, respectively the coding strand Mnl I restriction endonuclease sequence from normal and mutant Factor V nucleic acid. Another preferred oligonucleotide is that shown in SEQ ID NOs 23 and 25, respectively the coding strand Hind III restriction endonuclease sequence from normal and mutant Factor V nucleic acid.

Techniques for the separation of single stranded oligonucleotide, such as non-hybridized labeled oligonucleotide probe, from DNA duplex are well known, and typically involve the separation of single stranded from double stranded nucleic acids on the basis of their chemical properties. More often separation techniques involve the use of a heterogeneous hybridization format in which the non-hybridized probe is separated, typically by washing, from the DNA duplex that is bound to an insoluble matrix. Exemplary is the Southern blot technique, in which the matrix is a nitrocellulose sheet and the label is ³²p (Southern, J. Mol. Biol. 98:503, 1975).

The oligonucleotides can also be advantageously linked, typically at or near their 5′-terminus, to a solid matrix, i.e., aqueous insoluble solid support. Useful solid matrices are well known in the art and include cross-linked dextran such as that available under the tradename SEPHADEX from Pharmacia Fine Chemicals (Piscataway, N.J.); agarose, polystyrene or latex beads about 1 micron to about 5 mm in diameter, polyvinyl chloride, polystyrene, cross-linked polyacrylamide, nitrocellulose or nylon-based webs such as sheets, strips, paddles, plates microtiter plate wells and the like.

It is also possible to add “linking” nucleotides to the 5′ or 3′ end of the member oligonucleotide, and use the linking oligonucleotide to operatively link the member to the solid support.

In nucleotide hybridizing assays, the hybridization reaction mixture is maintained in the contemplated method under hybridizing conditions for a time period sufficient for the oligonucleotides having complementarity to the predetermined sequence on the template to hybridize to complementary nucleic acid sequences present in the template to form a hybridization product, i.e., a complex containing oligonucleotide and target nucleic acid.

The term hybridizing and phrase “hybridizing conditions” and their grammatical equivalents, when used with a maintenance time period, indicates subjecting the hybridization reaction admixture, in the context of the concentrations of reactants and accompanying reagents in the admixture, to time, temperature and pH conditions sufficient to allow one or more oligonucleotides to anneal with the target sequence, to form a nucleic acid duplex. Such time, temperature and pH conditions required to accomplish hybridization depend, as is well known in the art, on the length of the oligonucleotide to be hybridized, the degree of complementarity between the oligonucleotide and the target, the guanidine and cytosine content of the oligonucleotide, the stringency of hybridization desired, and the presence of salts or additional reagents in the hybridization reaction admixture as may affect the kinetics of hybridization. Methods for optimizing hybridization conditions for a given hybridization reaction admixture are well known in the art.

Typical hybridizing conditions include the use of solutions buffered to pH values between 4 and 9, and are carried out at temperatures from 4 degrees C (4° C.) to 37° C., preferably about 12° C. to about 30° C., more preferably about 22° C., and for time periods from 0.5 seconds to 24 hours, preferably 2 minutes (min) to 1 hour.

Hybridization can be carried out in a homogeneous or heterogeneous format as is well known. The homogeneous hybridization reaction occurs entirely in solution, in which both the oligonucleotide and the nucleic acid sequences to be hybridized (target) are present in soluble forms in solution. A heterogeneous reaction involves the use of a matrix that is insoluble in the reaction medium to which either the oligonucleotide, polynucleotide probe or target nucleic acid is bound.

Where the nucleic acid containing a target sequence is in a double-stranded (ds) form, it is preferred to first denature the dsDNA, as by heating or alkali treatment, prior to conducting the hybridization reaction. The denaturation of the dsDNA can be carried out prior to admixture with a oligonucleotide to be hybridized, or can be carried out after the admixture of the dsDNA with the oligonucleotide.

Predetermined complementarity between the oligonucleotide and the template is achieved in two alternative manners. A sequence in the template DNA may be known, such as where the primer to be formed can hybridize to known Factor V sequences and initiates primer extension into a region of DNA for subsequent assaying purposes as described herein, or where previous sequencing has determined a region of nucleotide sequence and the primer is designed to extend from the recently sequenced region into a region of unknown sequence.

Effective amounts of the oligonucleotide present in the hybridization reaction admixture are generally well known and are typically expressed in terms of molar ratios between the oligonucleotide to be hybridized and the template. Preferred ratios are hybridization reaction mixtures containing equimolar amounts of the target sequence and the oligonucleotide. As is well known, deviations from equal molarity will produce hybridization reaction products, although at lower efficiency. Thus, although ratios where one component can be in as much as 100-fold molar excess relative to the other component, excesses of less than 50-fold, preferably less than 10-fold, and more preferably less the 2-fold are desirable in practicing the invention.

(1) Detection of Membrane-immobilized Target Sequences

In the DNA (Southern) blot technique specific regions of genomic DNA are detected by immobilizing the target sequences on a membrane. The specific regions of genomic DNA are prepared by either PCR amplification, by PCR amplification followed by digestion with restriction endonucleases or by digestion with a restriction endonuclease without PCR amplification. Genomic DNA is first isolated. Specific regions of the genomic DNA are then PCR amplified to generate target sequences that are then analyzed intact or subjected to restriction digestion. Alternatively, the genomic DNA is cleaved by restriction endonucleases to form DNA fragments of discrete molecular weights.

The above-generated target sequences (DNA fragments) are then separated according to size in an agarose gel and transferred (blotted) onto a nitrocellulose or nylon membrane support. Conventional electrophoresis separates fragments ranging from 100 to 30,000 base pairs while pulsed field gel electrophoresis resolves fragments up to 20 million base pairs in length. The location on the membrane a containing particular target sequence is then determined by direct visualization of stained DNA. In other aspects, the sequence migration is determined by hybridization with a specific, labeled nucleic acid probe.

In alternative embodiments, target sequences are directly immobilized onto a solid-matrix (nitrocellulose membrane) using a dot-blot (slot-blot) apparatus, and analyzed by probe-hybridization. See U.S. Pat. Nos. 4,582,789 and 4,617,261.

Immobilized target sequences may be analyzed by probing with allele-specific oligonucleotide (ASO) probes, which are synthetic DNA oligomers of approximately 20 nucleotides, preferably 17 nucleotides in length. These probes are long enough to represent unique sequences in the genome, but sufficiently short to be destabilized by an internal mismatch in their hybridization to a target molecule. Thus, any sequences differing at single nucleotides may be distinguished by the different denaturation behaviors of hybrids between the ASO probe and normal or mutant targets under carefully controlled hybridization conditions.

(2) Detection of Target Sequences in Solution

Several rapid techniques that do not require nucleic acid purification or immobilization have been developed. For example, probe/target hybrids may be selectively isolated on a solid matrix, such as hydroxylapatite, which preferentially binds double-stranded nucleic acids. Alternatively, probe nucleic acids may be immobilized on a solid support and used to capture target sequences from solution. Detection of the target sequences can be accomplished with the aid of a second, labeled probe that is either displaced from the support by the target sequence in a competition-type assay or joined to the support via the bridging action of the target sequence in a sandwich-type format.

In the oligonucleotide ligation assay (OLA), the enzyme DNA ligase is used to covalently join two synthetic oligonucleotide sequences selected so that they can base pair with a target sequence in exact head-to-tail juxtaposition. Ligation of the two oligomers is prevented by the presence of mismatched nucleotides at the junction region. This procedure allows for the distinction between known sequence variants in samples of cells without the need for DNA purification. The joint of the two oligonucleotides may be monitored by immobilizing one of the two oligonucleotides and observing whether the second, labeled oligonucleotide is also captured.

(3) Scanning Techniques for Detection of Base Substitutions

Three techniques permit the analysis of probe/target duplexes several hundred base pairs in length for unknown single-nucleotide substitutions or other sequence differences. In the ribonuclease (RNase) A technique, the enzyme cleaves a labeled RNA probe at positions where it is mismatched to a target RNA or DNA sequence. The fragments may be separated according to size and the approximate position of the mutation identified. See U.S. Pat. No. 4,946,773.

In the denaturing gradient gel technique, a probe-target DNA duplex is analyzed by electrophoresis in a denaturing gradient of increasing strength. Denaturation is accompanied by a decrease in migration rate. A duplex with a mismatched base pair denatures more rapidly than a perfectly matched duplex.

A third method relies on chemical cleavage of mismatched base pairs. A mismatch between T and C, G, or T, as well as mismatches between C and T, A, or C, can be detected in heteroduplexes. Reaction with osmium tetroxide (T and C mismatches) or hydroxylamine (C mismatches) followed by treatment with piperidine cleaves the probe at the appropriate mismatch.

C. Compositions

The present invention also provides compositions of isolated polynucleotide sequences derived from a Factor V gene having a genetic mutation at nucleotide position 205 in exon 10.

As defined herein for compositions of this invention, the mutation identified at position 205 in exon 10 encompasses the corresponding Factor V cDNA sequence containing nucleotide position 1691. The genetic mutation as contemplated is a substitution of an adenine nucleotide for a guanine nucleotide.

The terms polynucleotide and polynucleotide sequence have been previously defined in Section B1.

The compositions as described herein are obtained by conventional nucleic acid procedures, including synthesis, isolation, purification, PCR amplification and the like. Particularly preferred are procedures including those specified for use with the methods of this invention described above that involve PCR amplification of a provided Factor V nucleic acid sample to produce an amplification product containing the polynucleotide sequence compositions as described herein.

In a preferred embodiment, an isolated polynucleotide sequence, derived from a Factor V gene having a genetic mutation at nucleotide position 205 in exon 10, comprises a nucleotide sequence from about 40 nucleotides to 6909 nucleotides in length. Preferred polynucleotide sequences within this preferred composition include those containing nucleotide sequences shown in SEQ ID NOs 7 and 23, or fragments thereof. The specific sequences have been previously described in Section B2a.

Also contemplated are compositions of preferred mutant polynucleotide sequences consisting essentially of a genomic DNA nucleotide sequences shown in SEQ ID NOs 3, 19, along with cDNA nucleotide sequences shown in SEQ ID NOs 28 and 26, the latter from nucleotide position 9 to nucleotide position 6917. The specific sequences have also been previously described in Sections B2a and B2b.

Another contemplated composition of the present invention are polynucleotide primers for use in amplifying a Factor V nucleic acid to produce an amplified product of this invention. The term primer has been previously defined in Section B1.

A preferred polynucleotide primer has the nucleotide sequence shown in SEQ ID NO 24 from nucleotide position 25 to nucleotide position 30. The preferred primer is capable of producing an amplification product containing a Hind III restriction endonuclease site in a Factor V gene having a guanine to adenine point mutation at nucleotide position 205 of exon 10. A particularly preferred polynucleotide primer consists essentially of a nucleotide sequence shown in SEQ ID NO 24.

D. Diagnostic Kits

The present invention also contemplates a diagnostic system, preferably in kit form, useful for the detection of a genetic mutation in a Factor V gene, associated with activated Protein C resistance, in a patient genomic DNA or mRNA nucleic acid sample according to the diagnostic methods and compositions described above. Thus, the diagnostic kits are useful for screening genomic DNA and cDNA as practiced in the methods of this invention.

The kit comprises, in an amount sufficient to perform at least one assay, a pair of primers comprising a first primer and a second primer capable of producing by PCR an amplification product that contains nucleotide position 205 of the Factor V gene. As defined herein, the mutation at this position contemplates a point mutation of a guanine nucleotide to an adenine nucleotide. As designated herein, the position of the mutation referred to by the position in exon 10 of Factor V genomic DNA encompasses the corresponding location in the Factor V cDNA.

In one aspect of the diagnostic kit, primers are in separate containers. In another aspect, the primer pair is retained within the same container. The polynucleotide primers contained within the kit are capable of amplifying a DNA product from a provided nucleic acid sample. The primers are thus designed for the amplification of a preselected region of nucleic acid sequence to allow for the detection of the presence or absence of the genetic point mutation in the Factor V gene associated with activated Protein C resistance. Preferred are primer pairs for amplifying both Factor V genomic DNA and cDNA. Particularly preferred primer pairs for amplifying genomic DNA include a first primer and a second primer having the paired nucleotide sequences SEQ ID NOs 4 and 5, and SEQ ID NOs 4 and 24. A particularly preferred primer for amplifying cDNA is the primer pair having the sequences shown in SEQ ID NOs 10 and 11.

In a further embodiment, the diagnostic kit further comprises a control polynucleotide sequence derived from a normal Factor V gene having a nucleotide sequence shown in SEQ ID NOs 2, 18 and 27. The term control indicates that the polynucleotide fragment having the noted sequence is provided in the kit as a standard that allows the practictioner a means to compare the restriction digestion patterns of the test or sample nucleic acid with that of the provided standard. As a result, the practictioner can verify that the amplified test product is comparable to that provided in the kit and that it is comparably digested producing equivalent restriction digestion products. The normal control polynucleotide sequences thus are included to provide a comparison with the patient's test samples for a means to determine the presence or absence of the point mutation of this invention in a patient sample. The preferred normal control polynucleotide sequences and their respective restriction endonuclease specificities have been previously described in Section B2.

The diagnostic kit of this invention further comprises a control polynucleotide sequence derived from a Factor V gene having a genetic mutation at nucleotide position 205 in exon 10. Preferred control mutated polynucleotide sequences include those shown in SEQ ID NOs 3, 19 and 28. As with the above normal control sequences, the mutant control polynucleotide sequences are included to provide a comparison with the patient's test samples for a means to determine the presence or absence of the point mutation of this invention in a patient sample. The preferred normal control polynucleotide sequences and their respective restriction endonuclease specificities have been previously described in Section B2.

Instructions for use of the packaged reagent are also typically included.

“Instructions for use” typically include a tangible expression describing the reagent concentration or at least one assay method parameter such as the relative amounts of reagent and sample to be admixed, maintenance time periods for reagent/sample admixtures, use of control polynucleotide sequences, temperature, buffer conditions and the like.

In one aspect of providing a diagnostic kit, the polynucleotide primers may be labeled with a detectable label. Radioactive elements are useful labeling agents and may be useful herein. An exemplary radiolabeling agent is a radioactive element that produces alpha ray emissions. Elements which themselves emit alpha rays, such as ³²p, ³⁵S, and ³³P represent one class of alpha ray emission-producing radioactive element indicating groups. Particularly preferred is ³²p Also useful is a beta emitter, such ¹¹¹indium or ³H.

The reagent species, polynucleotide or amplifying agent of any diagnostic system described herein can be provided in solution, as a liquid dispersion or as a substantially dry power, e.g., in lyophilized form.

The packaging materials discussed herein in relation to diagnostic systems are those customarily utilized in diagnostic systems.

The term “package” refers to a solid matrix or material such as glass, plastic (e.g., polyethylene, polypropylene and polycarbonate), paper, foil and the like capable of holding within fixed limits a diagnostic reagent such as a polynucleotide of the present invention. Thus, for example, a package can be a bottle, vial, plastic and plastic-foil laminated envelope or the like container used to contain a contemplated diagnostic reagent.

The materials for use in the assay of this invention are ideally suited for the preparation of a kit having sufficient amounts of materials to perform at least one assay. Such a kit may comprise a carrier means being compartmentalized to receive in close confinement one or more container means such as vials, tubes, and the like, each of the container means comprising one of the separate elements to be used in the method. For example, one of the container means may comprise a polynucleotide of the invention which is, or can be, detectably labeled. The kit may also have containers containing any of the other above-recited polynucleotide reagents used to practice the diagnostic methods.

### EXAMPLES

The following examples are intended to illustrate but are not to be construed as limiting of the specification and claims in any way.

1. Detection of the Guanine to Adenine Point Mutation in Genomic DNA From APC Resistant Family Patients

To determine the genetic basis for APC resistance in both unrelated and related patients, the nucleotide sequence of the Factor V gene was determined as described below. In addition, screening methods were developed to provide for alternative assays to determine the genetic basis for APC resistance in a patient. The methods used in making such a determination are described herein that allow detection of both the homozygous and heterozygous genotypes related to APC resistance.

A. Preparation and Nucleotide Sequence Determination of Genomic DNA

High molecular weight DNA was extracted from white blood cells from members of a family in which three sons had sustained recurrent venous thrombosis starting at an early age. The diagnosis of deep venous thrombosis (DVT) was well-established in the three patients, as was APC resistance in two of them, by anticoagulant response to APC as measured in the APTT clotting assay.

The APC ratios in two sons with recurrent thrombosis and two, as yet, asymptomatic daughters were less than or equal to 1.2. Normal APC ratios are ≧2.19 in males and ≧1.94 in females. The mother, who had sustained a DVT in association with her last pregnancy, and father had respective APC resistance ratios of 1.6 and 2.0. The levels of protein C, protein S, and antithrombin III were within normal limits in these individuals. One of the sons was not tested for APC resistance as he was chronically maintained on an oral anticoagulant. The use of oral anticoagulants, such as warfarin, interferes with the ability to obtain an accurate determination of APC resistance using the initially described APTT tests.

As previously discussed in Section B and as analyzed herein, in the heterozygous allelic genotype, the mutant allele has a point mutation characterized as a change in the nucleotide at nucleotide position 205 in exon 10 of the Factor V gene from a guanine to an adenine. The convention adopted for referring to the mutation site in exon 10 as nucleotide position 205 has been discussed in Section B. Also present in the heterozygous genotype is a normal allele which has a normal guanine nucleotide at the same nucleotide position.

In the homozygous mutant genotype, both alleles are mutant. In other words, both alleles have a change in the nucleotide from a guanine to an adenine at nucleotide position 205 in exon 10 of the Factor V gene.

In the homozygous normal genotype, both alleles are normal. In other words, both alleles have a guanine at nucleotide position 205 in exon 10 of the Factor V gene.

Therefore, to assay for the presence or absence of the normal and mutant alleles at nucleotide position 205 in exon 10 of the Factor V gene, based on a determination of nucleotide sequence as described above, blood was collected from APC resistance family members, anticoagulated with ACD anticoagulant and processed within 24 hours. In family members who had sustained venous thrombosis, samples were obtained at least three months after the most recent thrombotic episode. The pelleted erythrocytes, mononuclear and polynuclear cells were resuspended and diluted with a volume equal to the starting blood volume with chilled 0.14 M PBS, pH 7.4. The peripheral blood white blood cells were recovered from the diluted cell suspension by centrifugation on low endotoxin Ficoll-Hypaque (Sigma, St. Louis, Mo.) at 400×g for 10 minutes at 18° C. The pelleted white blood cells were then resuspended and used for the source of high molecular weight DNA.

The primers used in practicing this invention were synthesized on an Applied Biosystems 381A DNA Synthesizer following the manufacturer's instructions.

Genomic DNA was isolated from the peripheral blood samples as described by Lindblom, et al, Gene Anal. Tech., 5:97, 1988. Two μl out of the 50 μl genomic DNA were then diluted with 40 μl of a PCR reaction admixture containing 100 picomoles of the 5′ sense primer (also referred to as a first primer), FV7, having the sequence 5′-CATACTACAGTGACGTGGAC-3′ (SEQ ID NO 4), 100 picomoles of the 3′ anti-sense primer (also referred to as a second primer), FV8A, having the sequence 5′-TGCTGTTCGATGTCTGCTGC-3′ (SEQ ID NO 12), and a final concentration of 200 nM each of DATP, dCTP, dGTP, and dTTP, 1×Taq polymerase buffer (Promega, Madison, Wis.), 2 mM MgCl₂, and 0.5 units of Taq polymerase (Promega, Madison, WI).

The 5′ sense primer, FV7, corresponded to the nucleotide positions 115 through 134 in exon 10 of the Factor V gene, as shown in FIG. 5A (SEQ ID NO 15). The 3′ primer, FV8A, corresponded to the nucleotide positions 4 through 23 of Factor V exon 11. The reaction mixture was overlaid with mineral oil and subjected to 30 cycles of amplification. Each amplification cycle included denaturation at 94° C. for 1 minute, annealing at 60° C. for 2 minutes, and elongation at 72° C. for 3 minutes followed by 2 cycles of annealing at 60° C. for 2 minutes and elongation at 72° C. for 3 minutes. The amplification primers were removed from the reaction mixture prior to nucleotide sequence determination using Wizard PCR Prep columns according to the manufacturer's conditions (Promega, Madison, Wis.).

The resultant amplification products were comprised of a portion of the genomic DNA which corresponded to nucleotide positions 115 through 215 of exon 10, nucleotide positions 1 through ˜3100 of intron 10, and nucleotide positions 1 through 23 of exon 11 of the Factor V gene (Cripe et al, Biochem. 31:3777, 1992). The length of the amplified genomic DNA product was approximately 3200 base pairs.

To determine the nucleotide sequence of the resultant amplified DNA, sequencing reactions incorporating ³⁵S-DATP (Amersham, Arlington Heights, Ill.) were performed without further template purification using the fmol Cycle Sequencing Kit (Promega, Madison, Wis.) and the factor V-specific primer FV23 (5′-ATCGCCTCTGGGCTAATAGG-3′, SEQ ID NO 22). The FV23 primer corresponded to nucleotide positions 147 through 166 in exon 10 of the Factor V gene (FIG. 5A and SEQ ID NO 15).

The nucleotide sequences determined directly from the amplification products derived from the Factor V gene of the two patients with APC resistance showed one of two abnormalities. First, two bands were found in the sequencing gels which corresponded to nucleotide position 205 in exon 10 of the Factor V gene indicating that both the normal guanine nucleotide and the abnormal adenine nucleotide were at nucleotide position 205 in exon 10 of Factor V genomic DNA. Thus, a heterozygous allelic state for the point mutation was confirmed for one patient. Second, a single band was found in the sequencing gels which corresponded to nucleotide position 205 in exon 10 of the Factor V genomic DNA where only the abnormal adenine nucleotide was found. Thus, a homozygous mutant allelic state for the point mutation was also confirmed for the other patient.

The results of the nucleotide sequence determination of the Factor V genomic DNA amplification products prepared above revealed that the mutant allele containing the point mutation from a guanine to an adenine at nucleotide position 205 in exon 10 of the Factor V gene was otherwise identical to the normal allele.

B. Analysis of Restriction Digestion Fragments from Mnl I Incubated with Amplified Factor V Genomic DNA to Determine the Presence of the APC Resistance Point Mutation

Amplification products of shorter lengths containing nucleotide position 205 of exon 10 were generated as described in Example 1A with a different primer pair. As described herein, these smaller products were then subjected to digestion with Mnl I to allow for a determination of the presence or absence of the guanine to adenine point mutation at nucleotide position 205 of exon 10.

The APC resistance genotype is also characterized by a restriction polymorphism with the restriction endonuclease, Mnl I, in exon 10 of the Factor V gene. Normal alleles with a guanine at nucleotide position 205 in exon 10 of the Factor V gene contain the restriction endonuclease site for the restriction endonuclease Mnl I. Mutant alleles where a guanine has been changed to an adenine at nucleotide position 205 in exon 10 of the Factor V gene do not contain the restriction endonuclease site for the restriction endonuclease Mnl I. Nucleotide position 205 in exon 10 of the Factor V gene is contained within the nucleotide sequence of the Mnl I restriction site.

The presence or absence of this polymorphism can be detected by incubation of DNA containing the region bordering nucleotide position 205 in exon 10 of the Factor V gene in the presence of the restriction endonuclease Mnl I.

Mnl I is a type I restriction endonuclease which recognizes a specific double-stranded nucleotide sequence (restriction endonuclease recognition site or recognition site) and cleaves both strands of the double-stranded DNA at a position within the nucleotide sequence that is not contained within the recognition site.

Many type I restriction endonucleases cleave the double-stranded DNA at nucleotide positions which are random and thus generate restriction products containing a random number of nucleotides (Kleid et al, Proc. Natl. Acad. Sci. USA 73:293, 1976; Vissel et al, Nucl. Acids. Res. 16:4731, 1988). However, Mnl I has been shown to cleave the double-stranded DNA at nucleotide positions that are specific (Brinkley et al, Gene 100:267, 1991). Thus, Mnl I restriction products contain a specific number of nucleotides.

The recognition sites of the more commonly used type II restriction endonucleases are palindromic in contrast to type I restriction endonucleases that are not palindromic.

By convention, the recognition site for Mnl I is represented by the double-stranded nucleotide sequence as follows:

5′-CCTCNNNNNNN-3′ (SEQ ID NO 20)

3′-GGAGNNNNNN-5′ (SEQ ID NO 21)

wherein N is any nucleotide (Brinkley et al, Gene 100:267, 1991).

When the recognition site for Mnl I is inverted, it is represented by the double-stranded nucleotide sequence as follows:

5′-NNNNNNGAGG-3′ (SEQ ID NO 21)

3′-NNNNNNNCTCC-5′ (SEQ ID NO 20)

wherein N represents any nucleotide.

Thus, the coding strand nucleotide sequence 5′-ACAGGCGAGG-3′ (SEQ ID NO 6) in exon 10 of the Factor V gene from nucleotide positions 199 to 208 as shown in FIG. 5A (SEQ ID NO 15) and in the cDNA of Factor V cDNA from nucleotide positions 1685 to 1694 (FIGS. 1A-1C and SEQ ID NO 13) corresponds to the inverted Mnl I recognition sequence as shown in SEQ ID NO 21. As a result, the cleavage pattern by Mnl I results in a 3′ one base pair overhang as shown above.

The convention therefore adopted for determining the fragment sizes is based upon the Mnl I restriction digestion cleavage of the coding or sense strand of the amplification products which may have either of the above nucleotide sequence specificities. For example, as discussed below with either amplified genomic or cDNA from Factor V, the 5′ Mnl I site has the 3′ one-base overhang in the coding strand while the 3′ Mnl I site that contains nucleotide 205 in exon 10 (nucleotide 1691 in cDNA) is just the reverse having the 3′ one-base overhang in the noncoding or anti-sense strand. The coding strand of the 3′ Mnl I site therefore is the shorter cleavage product. Calculation of the restriction products resulting from Mnl I digestion based on the coding strand cleavage sites is either one base plus or minus depending on the double-stranded Mnl I site.

The sequence present in normal Factor V DNA provides a double-stranded DNA representing a Mnl I recognition site requiring a guanine nucleotide at nucleotide position 205 in exon 10 and 1691 in the cDNA sequence.

As a result, as shown below, an allele which contains the Mnl I recognition site at nucleotide positions 199 to 208 in exon 10 of the Factor V gene is a normal allele. Amplification products derived from the Factor V gene of a normal allele are cleaved in the presence of Mnl I to form restriction products.

A change in the nucleotide sequence representing a Mnl I recognition site from a guanine to an adenine in exon 10 of the Factor V gene at nucleotide position 205 and in the Factor V cDNA at nucleotide position 1691 therefore eliminates the Mnl I recognition site. As a result, an allele which does not contain a Mnl I recognition site at nucleotide positions 199 to 208 in exon 10 of the Factor V gene is a mutant allele. FIG. 5B (SEQ ID NO 16) shown the nucleotide sequence of exon 10 of such a mutant allele. Amplification products derived from the Factor V gene of a mutant allele which do not contain a Mnl I recognition site at nucleotide positions 199 to 208 in exon 10 of the Factor V gene are therefore not cleaved at that site in the presence of Mnl I.

In the heterozygous genotype, the presence of both the normal and mutant alleles are detected by the respective presence of both cleaved and uncleaved DNA upon incubation of amplification products derived from the Factor V gene of the normal and mutant alleles in the presence of the restriction endonuclease Mnl I.

Similarly, in the homozygous mutant genotype where both alleles are mutant, the restriction endonuclease Mnl I does not cleave at position 205 in exon 10 of the Factor V gene. Therefore, a homozygous mutant genotype can be detected by the lack of a Mnl I restriction endonuclease site containing nucleotide position 205 in exon 10 in amplification products derived from the Factor V gene of a mutant allele.

As discussed in Example 1A, the primer pair previously described amplified a nucleotide region having a Mnl I site naturally present in a normal allele.

For amplifying the smaller amplification products for subsequent restriction digestion analysis, the 51 sense primer, FV7 (SEQ ID NO 4), as described in Example 1A was paired with the 3′ anti-sense primer, FVINT102,) having the nucleotide sequence 5′-TGTTCTCTTGAAGGAAATGC-3′ (SEQ ID NO 5). The 5′ primer, FV7, corresponded to the nucleotide positions 115 through 134 in exon 10 of a Factor V gene (FIGS. 2A and 2B). The 3′ primer, FVINT102, corresponded to the nucleotide positions 86 through 105-of the Factor V intron 10.

Genomic DNA was isolated from family members as described in Example 1A. Two μl of the 50 μl of isolated genomic DNA was then diluted with 50 μl of a PCR reaction admixture containing 100 picomoles of the 5′ sense primer, FV7, 100 picomoles of the 3′ anti-sense primer, FVINT102, and a final concentration of 200 nM each of DATP, dCTP, dGTP, and dTTP, 1×Taq polymerase buffer (Promega, Madison, Wis.), 1.5 mM MgCl₂, and 0.5 units of Taq polymerase (Promega, Madison, Wis.). The reaction mixture was overlaid with mineral oil and subjected to 30 cycles of amplification. Each amplification cycle included denaturation at 94° C. for 1 minute, annealing at 60° C. for 2 minutes, and elongation at 72° C. for 2 minutes. Two additional cycles of annealing at 60° C. for 2 minutes and amplification at 72° C. for 3 minutes were also performed.

The normal and mutant nucleotide sequence of the coding strand of the resultant amplification products generated by PCR amplification of genomic DNA with the primers FV7 and FVINT102 is given respectively in FIGS. 2A and 2B. The amplification products of the normal and mutant alleles were 206 base pairs in length.

Ten μl of the 50 μl amplification products derived from a Factor V gene of a normal or mutant allele were then maintained in a 20 μl digestion system with 1×buffer number 2 (New England Biolabs, Beverly, Mass.), 1.65×bovine serum albumin (New England Biolabs, Beverly, Mass.), and 1.7 units Mnl I restriction endonuclease (New England Biolabs, Beverly, Mass.) for 2 hours at 37° C. Ten μl of the digestion products were admixed with gel loading dye buffer and separated according to molecular weight by electrophoresis on an agarose gel consisting of 3% (w/v) NuSieve agarose (FMC) with 0.5% (w/v) agarose.

Additionally, the primers FV7 and FVINT102 for amplifying the region of DNA having nucleotide position 205 of the Factor V gene were also designed to amplify a region of genomic DNA which includes a second Mnl I restriction endonuclease site at nucleotide positions 152 to 162 in exon 10, shown in FIG. 2A, of the Factor V gene. As previously defined, this second Mnl I restriction site is referred to as the 5′ Mnl I site as it is located 5′ to the Mnl I site containing nucleotide position 205. Accordingly, the latter site is referred to as the 3′ site. The presence of a second Mnl I restriction endonuclease site in the amplification products derived from a Factor V gene of both the normal and mutant alleles provides a control for verifying that amplification products prepared as described herein are capable of being cleaved when incubated in the presence of the restriction enzyme Mnl I to form restriction products.

The results of Mnl I restriction digestion of genomic DNA amplified products are shown in FIG. 4. A photograph of an agarose gel contains DNA representing a portion of genomic DNA including nucleotide position 205 in exon 10 of the Factor V gene which has been incubated in the presence of the restriction endonuclease Mnl I and separated electrophoretically. Lane 1 contains DNA molecular weight markers as indicated in base pairs (bp). Lanes 2, 4, 5, and 6 contain amplified genomic DNA isolated from APC resistance patients that are heterozygous for the point mutation at nucleotide position 205 in exon 10 of the Factor V gene. Lane 3 contains amplified genomic DNA isolated from a normal patient that is homozygous for the normal or nonmutant allele.

In a homozygous state where both alleles are normal alleles and do not contain the point mutation at nucleotide position 205 in exon 10 of a Factor V gene, the amplification products derived from a Factor V gene of the normal alleles were cleaved at the Mnl I restriction endonuclease site which contains nucleotide position 205 in exon 10 of the Factor V gene. The amplification products derived from a Factor V gene of the normal allele were also cleaved at the other native Mnl I restriction endonuclease site as described above.

The cleavage of the 206 base pair amplification products derived from a Factor V gene of a normal allele (FIG. 2A and SEQ ID NO 2) at both of the Mnl I restriction endonuclease sites resulted in three restriction products 37, 47, and 122 base pairs each as shown in FIG. 4, lane 3. Since the cleavage pattern by Mnl I results in a 3′ one base pair overhang as previously discussed, the convention therefore adopted for determining the fragment sizes is based upon the Mnl I restriction digestion cleavage of the coding or sense strand of the amplification products.

Therefore, digestion at the 5′ Mnl I site, having the one-base 3′ overhang, of the amplification product resulted in the 47 base pair fragment. Digestion at the 3′ Mnl I, not having the one-base 3′ overhang, generated the 37 base pair fragment between the 5′ and 3′ Mnl I sites. The remaining fragment of 122 base pairs extends from the 3′ Mnl I site to the 3′ end of the amplication product. The three fragments combine to form the 206 base pair amplification product.

The two smallest fragments are not apparent in the reproduced figure as shown. However, the presence of three distinct bands were directly visualized in the original gel preparation indicating that both the first and second Mnl I restriction endonuclease sites were digested in the Factor V genomic DNA amplification products, the latter having a nucleotide sequence listed in SEQ ID NO 2 and shown in FIG. 2A.

Had the Mnl I site that is 5′ to the Mnl I site having nucleotide position 205 not been cleaved, two restriction fragment products would have resulted being 84 and 122 base pairs each. The 84 base pair fragment thus was cleaved in the above digestion to two fragments of 37 and 47 base pairs each reflecting the specificity of the Mnl I digestion.

In contrast to digestion of amplified products of a normal allele, Mnl I digestion of amplified products from a mutant allele resulted in only two restriction fragment products of 47 and 159 base pairs each. As previously discussed, the Mnl I site at nucleotide position 205 in exon 10 is destroyed if the guanine to adenine point mutation is present. Therefore, the mutant amplified product contains only the other Mnl I restriction site located 5′ to the destroyed site.

Cleavage and lack of cleavage of the amplification products derived from a Factor V gene of a normal or mutant allele was determined by visualizing the number and molecular weight of restriction products subsequent to incubation of the amplification products in the presence of the restriction enzyme Mnl I.

Without Mnl I digestion, the amplified mutant products, as shown in FIG. 2B and listed in SEQ ID NO 3 has restriction products totaling 206 base pairs. With Mnl I digestion of the 5′ Mnl I restriction site, cleavage of the mutant amplification product resulted in generating two restriction products of 159 and 47 base pairs. If the gel contained only these two fragments, a homozygous mutant allelic state would be confirmed.

Combining the separate restriction digestion patterns from above presents a profile of a heterozygous state having both a normal and a mutant allele as shown in FIG. 4 in lanes 2, 4, 5, and 6. The presence of restriction products of 37, 47, 122, and 159 base pairs each is indicative of the expected results for both the normal and mutant alleles confirming a heterozygous state. While the resolution of the 37 and 47 base pair restriction products may be difficult to visualize in the photograph of the agarose gel, they were observed in the original gel preparation. The 122 and 159 base pair bands are easily visualized in FIG. 4.

Thus, Mnl I restriction digestion analysis of PCR amplified products derived from a Factor V gene of a patient's genomic DNA provides for the ability to distinguish between the allelic states of homozygous normal, homozygous mutant and heterozygous.

C. Preparation of Amplification Products from Genomic DNA and Detection of a Hind III Restriction Site to Identify the Presence of a Guanine to Adenine Point Mutation

The presence of a point mutation in which a guanine nucleotide is changed to an adenine nucleotide at nucleotide position 205 in exon 10 of the Factor V gene is also detected by preparation of amplification products from genomic DNA and the subsequent detection of an engineered Hind III restriction site.

As described in Section B2, the Hind III restriction endonuclease site used in determining the presence or absence of the guanine to adenine point mutation is not present at that location in either the normal or mutant Factor V allele. Therefore, the Hind III primer is designed to take advantage of the adenine mutation present in a mutant allele with the concomitant introduction of three additional point mutations to create a Hind III site that is present in the mutant allele but absent from the normal counterpart.

Hind III is a type II restriction endonuclease which recognizes a specific double-stranded nucleotide sequence (restriction endonuclease recognition site or recognition site) and cleaves both strands of the double-stranded DNA at a position within the recognition site.

The type II restriction endonuclease Hind III (Hind III) recognizes double-stranded DNA represented by the following sequence:

5′-AAGCTT-3′ (SEQ ID NO 23).

Incubation of double-stranded DNA containing the sequence given in SEQ ID NO 23 in the presence of the restriction endonuclease Hind III results in the cleavage of both strands of the DNA to generate the following structure:

5′-A-3′

3′-TTCGA-5′ (SEQ ID NO 23, from nucleotide position 2-6 as shown in the 5′ to 3′ direction).

The specific cleavage of type II restriction endonucleases generates restriction products containing a specific number of nucleotides. Thus, Hind III restriction products containing a specific number of nucleotides are generated by cleavage of double-stranded DNA with Hind III.

The coding strand nucleotide sequence 5′-AAGCTT-3′ (SEQ ID NO 23) in the amplification products produced as described below of a mutant allele corresponds to nucleotide positions 205 to 210 in exon 10 of a Factor V gene as shown in FIG. 7B (SEQ ID NO 19). Thus, the sequence is the top or coding single strand of the double-stranded DNA representing a Hind III recognition site.

Amplification products derived from a Factor V gene of the mutant allele are cleaved at nucleotide position 205 when incubated in the presence of the restriction endonuclease Hind III to produce restriction products.

However, the nucleotide sequence 5′-GAGCTT-3′ (SEQ ID NO 25) in corresponding normal allelic amplification products occurring at the same location does not represent a Hind III recognition site. Amplification products derived from a Factor V gene of a normal allele thus are not cleaved at nucleotide position 205 when incubated in the presence of the restriction endonuclease Hind III.

The lack of cleavage of the amplification products derived from the Factor V gene of a normal allele results in a single DNA fragment which is detected by acrylamide gel electrophoresis as described herein.

Thus, in both heterozygotic and homozygotic genotypes, the mutant amplified alleles (FIG. 7B and SEQ ID NO 19) are Hind III digested to form two distinct restriction fragments as compared to the undigested fragment of the amplified normal counterparts (FIG. 7A and SEQ ID NO 18).

The primers for amplifying the region of DNA having the point mutation are designed to amplify a region of genomic DNA containing nucleotide positions 205 to 210 in exon 10 of the Factor V gene. The 3′ anti-sense primer, also referred to as a second primer, are designed to introduce a portion of the Hind III restriction endonuclease site into the amplification product derived from a Factor V gene, as the normal allele does not have a natural Hind III site in this region.

Therefore, to introduce a Hind III site into a mutant allele, the primer pairs FV7 (SEQ ID NO 4) and FV506tst2 (SEQ ID NO 24) are used in PCR as described before.

The 5′ sense or first primer, FV7, is described in Example 1A and corresponds to nucleotide positions 115 through 134 in exon 10 of the Factor V gene (FIGS. 7A and 7B). The 3′ anti-sense or second primer, FV506tst2, has the nucleotide sequence 5′-TTACTTCAAGGACAAAATACCTGTAAAGCT-3′ (SEQ ID NO 24). The 3′ primer corresponds to nucleotide positions 206 through 215 in exon 10 of the Factor V gene and nucleotide positions 1 through 20 in intron 10 of the Factor V gene (FIGS. 7A and 7B).

The 3′ primer also introduces three additional point mutations at sites corresponding to nucleotide positions 208-210 into the resultant amplification products derived from a Factor V gene. The additional point mutations are not normally present in either the normal allele or the mutant allele. These point mutations are therefore introduced into the amplification products to form a portion of the Hind III restriction endonuclease site (FIG. 7B) that relies upon the presence of the adenine point mutation at nucleotide position 205 in exon 10.

The adenine at nucleotide position 205 in exon 10 of a Factor V gene of a mutant allele along with a portion of the Hind III restriction endonuclease site introduced by the 3′ anti-sense primer forms a Hind III restriction endonuclease site (FIG. 7B).

Therefore, the amplification products derived from a Factor V gene of a mutant allele are cleaved when incubated in the presence of the Hind III restriction endonuclease.

In other words, the guanine at nucleotide position 205 in exon 10 of a Factor V gene of a normal allele along with a portion of the Hind III restriction endonuclease site introduced by the 3′ anti-sense primer does not form a Hind III restriction endonuclease site (FIG. 7A). Therefore, the amplification products derived from a Factor V gene of a normal allele are not cleaved when incubated in the presence of the Hind III restriction endonuclease.

The nucleotide sequences of the amplification products generated by PCR amplification with the primers FV7 and FV506tst2 of a normal and mutant allele are given in FIGS. 7A and 7B, respectively.

For the PCR amplification to introduce a Hind III site into a mutant allele, 2 μl of the 50 μl of isolated genomic DNA from patients as described in Example 1A are diluted with 50 μl of a PCR reaction admixture containing 100 picomoles of the 5′ sense primer, FV7, 100 picomoles of the 3′ anti-sense primer, FV506tst2, and a final concentration of 200 nM each of DATP, dCTP, dGTP, and dTTP, 1×Taq polymerase buffer (Promega, Madison, Wis.), 1.5 mM MgCl₂, and 0.5 units of Taq polymerase (Promega, Madison, Wis.). The reaction mixture is overlaid with mineral oil and subjected to 30 cycles of amplification. Each amplification cycle includes denaturation at 94° C. for 1 minute, annealing at 60° C. for 2 minutes, and elongation at 72° C. for 2 minutes. Two additional cycles of annealing at 60° C. for 2 minutes and amplification at 72° C. for 3 minutes are also performed.

PCR amplification of a Factor V gene of a normal or mutant allele with the primers FV7 and FV506tst2 results in an amplification product of 121 base pairs in length.

Ten μl of the resultant 50 μl amplification products derived from exon 10 and intron 10 of a Factor V gene of a normal or mutant allele are then maintained in a 20 μl digestion system with 1×buffer number 2 (New England Biolabs, Beverly, Mass.) and 2 units Hind III restriction endonuclease (New England Biolabs, Beverly, Mass.) for 2 hours at 37° C. Ten μl of the digestion products are admixed with gel loading dye buffer and separated according to molecular weight by acrylamide gel electrophoresis (Sambrook et al, Molecular Cloning, A Laboratory Manual, 2nd ed., Cold Spring Harbor Press, Cold Spring Harbor, N.Y., 1989).

The respective cleavage and lack of cleavage of the mutant and normal amplification products derived from a Factor V gene is thus determined by visualizing the number and molecular weight of restriction products subsequent to incubation of the amplification products in the presence of the restriction enzyme Hind III.

Amplification products derived from exon 10 and intron 10 of a Factor V gene of a normal allele do not contain the recognition site for the restriction endonuclease Hind III and therefore are not cleaved when incubated in the presence of the restriction enzyme Hind III. The lack of cleavage of the amplification products results in a single DNA fragment of 121 base pairs. Thus, the absence of the Hind III restriction endonuclease site provides a means of identifying a normal allele of a Factor V gene.

The amplification products derived from a Factor V gene of a mutant allele having an adenine nucleotide at nucleotide position 205 in exon 10 of the Factor V gene along with the other PCR-introduced point mutations at nucleotide positions 208-210 (5′-CTT-3′ for 5′-GAA-3′ of the coding or sense strand) are cleaved when incubated in the presence of the restriction enzyme Hind III. Cleavage of the amplification product derived from a Factor V gene of a mutant allele at the Hind III restriction endonuclease site results in two restriction products of 91 and 30 base pairs in length, based on the cleavage products generated on the coding or sense strand as was the convention for the Mnl I digestions described in Example 1B. Thus, the presence of the engineered Hind III restriction endonuclease site provides a means of identifying a Factor V gene of a mutant allele.

The use of PCR to generate amplification products derived from a Factor V gene of a normal or mutant allele to introduce a portion of a Hind III recognition site followed by the incubation of the amplification products in the presence of the Hind III restriction endonuclease is another means in addition to the first approach with Mnl I digestion as described in Example 1B for confirming the presence or absence of a point mutation at nucleotide position 205 in exon 10 of the Factor V gene, and thereby identify a patient's genetic basis for APC resistance.

D. PCR Amplification and Nucleotide Sequence Determination of Amplified cDNA Products

To determine the genetic basis of APC resistance in patients, the nucleotide sequence of Factor V cDNA from eight unrelated APC resistant patients was determined.

Standard polymerase chain reaction techniques followed by nucleotide sequence determination as described herein were used to identify genetic mutations in the Factor V cDNA, including the guanine to adenine point mutation at nucleotide position 1691.

Peripheral blood samples were obtained from eight consecutively identified unrelated APC resistant patients. Six of the eight patients were symptomatic and had an average age of 29 years. The six patients included the previously described patients BB and LS (Griffin et al, Blood 82:1989, 1993; Sun et al, Blood 83:3120, 1994). The two additional asymptomatic subjects were 46 and 62 years of age.

Total cellular RNA was purified from lymphoblasts from each of the patients described above. RNA was prepared from buffy coats of whole blood collected in ACD using RNA-Stat 60 following the manufacturer's recommended procedures (TelTest “B”, Friendswood, Tex.). No attempt was made to exclude platelets, as platelets contain RNA derived from the parent megakaryocytes. The isolated RNA was resuspended in water.

In preparation for PCR amplification, RNA was isolated as described above and used as a template for cDNA synthesis using the cDNA Cycle Kit (Invitrogen, San Diego, Calif.). In an 50 μl transcription reaction, 50-200 nanograms (ng) of RNA was first annealed with 250 ng of oligonucleotide d(T) (oligo-dT) primer. Single stranded cDNA was then synthesized in a first strand cDNA synthesis reaction using the cDNA Cycle Kit according to the manufacturer's recommended procedures. The resultant noncoding strand of cDNA was then used as a template for PCR amplification using Factor V-specific primers.

In the PCR amplification, the first primer (e.g., FV7) hybridized to the noncoding strand of cDNA at a position 3′ to nucleotide position 1691 of a Factor V gene and a primer extension reaction was initiated to generate an amplification product which corresponded to the coding strand of cDNA. The second primer (e.g., FV8A) hybridized to the amplified coding strand of cDNA at a position 3′ to nucleotide position 1691 of a Factor V gene and a primer extension was initiated to generate an amplification product which corresponded to the noncoding strand of cDNA.

In an alternative approach, the noncoding strand of cDNA is generated by the method described above in a first strand synthesis reaction and used as a template to generate the coding strand of cDNA in a second strand synthesis reaction by methods commonly used to generate cDNA libraries which are well known to those of skill in the art and are described in Sambrook et al, Molecular Cloning. A Laboratory Manual, 2nd ed., Cold Spring Harbor Press, Cold Spring Harbor, N.Y., 1989. The resultant first and second strands are then used as templates as the noncoding and coding strands of cDNA, respectively, in the subsequent amplification reactions.

The sequence encoding the Factor V light chain was amplified using the primers FV9 (5′-TGAGATCATTCCAAAGGAAG-3′, SEQ ID NO 8) and FV14 (5′-TTGAGGTCTTAAAGAGTCTC-3′, SEQ ID NO 9). The 5′ sense or coding primer corresponded to nucleotide positions 4659 through 4678 of Factor V cDNA. The 3′ anti-sense or noncoding primer corresponded to nucleotide positions 6792-6811 of SEQ ID NO 13 (the entire normal Factor V cDNA nucleotide sequence—see also FIGS. 1A-1C) in the 3′ untranslated region of Factor V cDNA following the translational stop codon.

The nucleotide sequence encoding the connecting region is located 5′ of the nucleotide sequence encoding the light chain.

Two μl of the synthesized cDNA were then diluted with 48 μl of a PCR reaction admixture containing 100 picomoles (pmol) of the 5′ sense primer FV9 and the 3′ anti-sense primer FV14 in a final concentration of 200 nM each of DATP, dCTP, dGTP, and dTTP, 1.5 mM MgCl₂, 1×Taq polymerase buffer (Promega, Madison, Wis.) and 0.5 U of Taq polymerase. The reaction mixture was overlaid with mineral oil and subjected to 30 cycles of amplification. Each amplification cycle included denaturation at 94° C. for 1 minute, annealing at 56° C. for 2 minutes and elongation at 72° C. for 3 minutes.

The resultant amplification product contained a portion of the sequence of the cDNA which corresponded to the 3′ region of the connecting region (nucleotide positions 4659 to 4808 as shown in SEQ ID NO 13 and FIGS. 1A-1C) and extended through the entire nucleotide sequence which corresponded to the light chain including the translational stop codon (nucleotide position 4809-6765 as shown in SEQ ID NO 13) and a small portion of the 3′ untranslated region (ending at nucleotide position 6811 of SEQ ID NO 13). The amplified products were approximately 2153 base pairs in length.

The sequence encoding the Factor V heavy chain was also amplified using the primer pairs, FV13 (5′-CAGGAAAGGAAGCATGTTCC-3′, SEQ ID NO 10) and FV2 (5′-TGCCATTCTCCAGAGCTAGG-3′, SEQ ID NO 11). The 5′ sense primer or first primer, FV13, corresponded to nucleotide positions 78 through 97 of Factor V cDNA shown in FIGS. 1A-1C (SEQ ID NO 13) within the 5′ untranslated region and the 5′ region of the nucleotide sequence which encodes the beginning of the heavy chain region (FIGS. 1A-1C and FIG. 6A). The nucleotide sequence encoding the heavy chain region is located 5′ of the nucleotide sequence encoding the connecting chain region. The 3′ anti-sense primer or second primer, FV2, corresponded to the nucleotide positions 2355 through 2374 in the heavy chain region of Factor V (FIGS. 1A-1C and FIG. 6A).

Two μl of the cDNA were then diluted with 48 μl of a PCR reaction admixture as described for the light chain amplification containing 100 picomoles (pmol) of the 5′ sense primer FV13 and the 3′ anti-sense primer FV2.

The resultant amplification products derived from the heavy chain region of the Factor V cDNA contained a portion of the Factor V cDNA which corresponded to nucleotide positions 78 through 90 of the 3′ untranslated region of the Factor V cDNA, extended through entire nucleotide sequence which corresponds to the heavy chain region (nucleotide positions 91 through 2301), and nucleotide positions 2302 through 2374 of the nucleotide sequence which corresponds to a portion of the connecting region. The normal and mutant amplification products were approximately 2297 base pairs in length as respectively shown in FIGS. 6A (SEQ ID NO 27) and 6B (SEQ ID NO 28).

The guanine or adenine nucleotide as shown corresponds to nucleotide position 1614 in the above figures and sequences. However, the position is in fact position 1691 in intact Factor V cDNA. The basis for the discrepancy in the nucleotide position of the guanine or adenine nucleotides being at 1614 in SEQ ID NOS 27 and 28 as compared to 1691 in intact Factor V cDNA stems from the Sequence Listing convention for numbering the amplified cDNA as shown in SEQ ID NOS 27 and 28. In the latter, nucleotide position 1 corresponds to nucleotide position 78 in Factor V cDNA as shown in SEQ ID NO 13 and in the both FIGS. 6A and 6B. As the 5′ primer, FV7, begins at nucleotide position 78 in intact cDNA, that position correspondingly becomes nucleotide position number 1 when the amplified sequence is separately presented in the Sequence Listing.

The above sequences have also been compiled into one sequence listed in SEQ ID NO 17 having a “N” nucleotide corresponding to the mutation site with N is either a guanine or an adenine, wherein the N is located at nucleotide position 1614 that corresponds to nucleotide position 1691 in intact Factor V cDNA.

To determine the nucleotide sequence of the resultant amplification products, sequencing reactions incorporating ³⁵S-DATP (Amersham, Arlington Heights, Ill.) were performed without further template purification. Several different primers whose sequence was based upon the nucleotide sequence of the Factor V cDNA (Jenny et al, Proc. Natl. Acad. Sci. USA 84:4846, 1987) in separate sequencing reactions were used to determine the nucleotide sequence of the amplification products using the fmol Cycle Sequencing Kit according to the manufacturer's recommended procedures (Promega, Madison, Wis.).

Three nucleotide differences were observed at nucleotide positions 1691, 6727, and 5380 of the normal Factor V cDNA shown in FIGS. 1A-1C (SEQ ID NO 13) which caused an alteration in the amino acid residue which was encoded at that nucleotide position. The convention for numbering of the cDNA sequence is that described by Jenny et al and is more thoroughly discussed in the figure legend for FIG. 1.

A point mutation at nucleotide position 1691 of the Factor V cDNA was identified by the presence of two bands in the sequencing gels. The two bands represented both the normal guanine nucleotide and the mutant adenine nucleotide at nucleotide position 1691 of the Factor V cDNA. Thus, a heterozygous allelic state for the point mutation was determined for this patient's Factor V gene. The point mutation at nucleotide position 1691 of Factor V cDNA is predicted to change the normally encoded amino acid residue at position 506 from an arginine to a glutamine.

Point mutations were also identified for at nucleotide positions 6727 and 5380 which affected the encoded amino acid. The normal nucleotide at position 6727, an adenine, was changed to a guanine thereby changing the normally encoded amino acid residue at position 2185 from a threonine to an alanine. The normal nucleotide at position 5380, a guanine, was changed to an adenine thereby changing the normally encoded amino acid residue at position 1736 from a valine to a methionine.

The nucleotide sequences of both the amplified normal and mutant cDNAs from amplification with the primer pair FV13 and FV2 are respectively shown in FIGS. 6A (SEQ ID NO 27) and 6B (SEQ ID NO 28).

Thus, determining the nucleotide sequence of the Factor V cDNA from patients having APC resistance identified, among two other mutations, a unique point mutation characterized as a change of a guanine nucleotide to an adenine nucleotide at nucleotide position 1691 of the Factor V cDNA. Nucleotide position 1691 of the Factor V cDNA corresponds to nucleotide position 205 in exon 10 of Factor V genomic DNA.

The nucleotide difference at nucleotide position 1691 of the Factor V cDNA and in the corresponding genomic DNA represents the genetic basis of APC resistance. The peptide bond between the amino acid residue arginine at position 506, encoded in part by nucleotide position 1691, and the amino acid residue glycine at position 507 a first bond cleaved by APC during inactivation of Factor Va. Therefore, the observed resistance of Factor Va variant, which contains the amino acid residue glutamine in place of the amino acid residue arginine at amino acid position 506 to cleavage by APC is easily rationalized on a biochemical level (Sun et al, Blood 83:3120, 1994). The dominant transmission of APC resistance is also readily understood (Dahlback et al, Proc. Natl. Acad. Sci. USA 90:1004, 1993; Svensson et al, N. Enal. J. Med. 330:517, 1994).

In contrast, the nucleotide differences at nucleotide positions 6727 and 5380 represent neutral changes in the encoded amino acid residue and do not result in APC resistance.

RNA was also isolated from related APC resistance patients, converted to cDNA, and amplified by PCR to provide a template for nucleotide sequence determination. The Factor V heavy chain region with a portion of the 5′ untranslated and connecting regions and the Factor V light chain region with a portion of the connecting and 3′ untranslated regions were amplified in separate reactions.

Total cellular RNA was purified as described above from lymphoblasts from each member of the family described above in Example 1A. RNA was converted to cDNA also as described above.

The nucleotide sequence encoding the Factor V light chain was amplified using the primers FV9 (SEQ ID NO 8) and FV14 (SEQ ID NO 9) as described above.

The nucleotide sequence encoding the heavy chain region of Factor V cDNA was also amplified as previously described using the primers FV13 (SEQ ID NO 10) and FV2 (SEQ ID NO 11). The nucleotide sequences of both the amplified normal and mutant cDNAs from amplification with the primer pair FV13 and FV2 are respectively listed in SEQ ID NOS 27 and 28 as previously described.

The nucleotide sequences determined directly from the resultant amplification products derived from the heavy chain region of Factor V cDNA from the family members having DVT showed one of two abnormalities. Two bands corresponding to nucleotide position 1691 of the cDNA 28 were found in the sequencing gels where both the normal guanine nucleotide and the abnormal adenine nucleotide were found (FIG. 3, sample II-3). Thus, a heterozygous state for the point mutation was determined. Secondly, a change of a guanine nucleotide to an adenine nucleotide which corresponded to nucleotide position 1691 of the cDNA was found (FIG. 3, sample II-2). Thus, a homozygous state for the point mutation was determined.

The nucleotide sequence determination of amplification products derived from Factor V cDNA of patients having APC resistance confirmed the presence of a unique point mutation. This point mutation was characterized as a change of a guanine nucleotide to an adenine nucleotide at nucleotide position 1691 of the Factor V cDNA.

E. PCR Amplification and Mnl I Restriction Analysis of Amplified cDNA Products

Another assay method, in addition to nucleotide sequencing as described in Example 1D, for determining the presence or absence of the guanine to adenine point mutation in amplified cDNA is restriction digestion analysis as previously described for genomic DNA in Examples 1B and 1C.

To obtain suitable cDNA amplification products for subsequent digestion with the restriction endonuclease Mnl I, the 2297 base pair heavy chain cDNA amplification products, that are either normal (SEQ ID NO 27) or mutant amplified cDNA (SEQ ID NO 28), resulting from PCR with the primer pair FV13 and FV2, as described in Example 1D, were used as templates in a second round of PCR as described herein.

For the second PCR, the PCR primer pair, the 5′ sense or first primer FV7 (SEQ ID NO 4) and the 3′ anti-sense or second primer FV8A (SEQ ID NO 12), both of which were used to amplify genomic DNA as described in Example 1A, were used in PCR with the above amplified cDNA templates corresponding to SEQ ID NOS 27 or 28. The PCR amplification was performed as previously described in Example 1D for the second round.

The normal and mutant cDNA amplification products formed from the second round of PCR have the respective 124 base pair nucleotide sequences shown in SEQ ID NOS 13 and 26, both from nucleotide position 1601 to 1724.

The resultant amplification products generated above were then subjected to restriction digestion with Mnl I as previously described for Factor V genomic DNA. As with genomic DNA restriction digestion analysis, the analysis of the differential restriction product fragments by electrophoresis provided the confirmation of the presence or absence of the guanine to adenine point mutation in a patient's mRNA sample.

With normal cDNA having a guanine nucleotide at nucleotide position 1691, the second round amplification products contained the two Mnl I restriction sites (the sites referred to as 5′ and 3′ to position 1691) as previously described for genomic DNA restriction analysis in Example 1B. In contrast, the corresponding mutant amplified cDNA only had the 5′ Mnl I site that did not contain the point mutation.

As such, the expected Mnl I restriction digestion products of the 124 base pair normal cDNA amplification products, if both Mnl I sites are cleaved properly, are three fragments of 47, 37 and 40 base pairs. In the 124 base pair mutant cDNA amplification products, since only the 5′ Mnl I is present, the expected restriction digestion products are two fragments of 47 and 77 base pairs. Accordingly, a heterozygotic pattern would contain 37, 40, 47 and 77 base pair fragments. Therefore, analysis of the Mnl I restriction digestion patterns from the normal and mutant 124 base pair amplification products from the cDNA allows for the determination of the presence or absence of the guanine to adenine point mutation at nucleotide position 1691 as well as the determination of the genotype.

In addition, nucleotide sequence analysis as previously described is also performed on the 124 base pair cDNA amplification products to confirm the presence or absence of the point mutation.

The above-described 124 base pair cDNA amplification products are also generated from a one-step PCR on the non-amplified cDNA template that was directly synthesized from mRNA as described in Example 1D. The PCR primer pair, FV7 and FV8A, are used on the intact cDNA template to produce the short cDNA amplification products. Restriction digestion analysis and/or nucleotide sequence determination assay methods are then performed on the resultant cDNA amplification products as described above.

In an alternative approach to amplifying cDNA, all or a part of the regions of Factor V cDNA comprising the 5′ untranslated region, heavy chain region, connecting region, light chain region, and 3′ untranslated region (SEQ ID NO 13 from nucleotide positions 9 to 6917) are amplified in a single reaction prior to nucleotide sequence determination. The amplification products resulting from amplification of the regions of Factor V cDNA are from about 40 to 6909 base pairs in length. More preferably, the amplification products contain the Factor V heavy chain region. An exemplary amplification product containing the point mutation at nucleotide position 1691 in the Factor V cDNA is given in SEQ ID NO 26 from nucleotide position 9 to nucleotide position 6917.

2. Conclusion

The molecular basis of the Factor V mutation and the resultant APC resistance has now been described by others following the discovery and generation of methods and compositions of this invention to detect the guanine to adenine point mutation in the Factor V gene associated with APC resistance as described herein. For example, Bertina et al, Nature 369:64, 1994 and Voorberg et al, Lancet 343:1535, 1994 identified the point mutation at nucleotide position 1691 in Factor V cDNA by PCR amplification of the Factor V cDNA followed by nucleotide sequence analysis.

The same point mutation has now been widely described in APC resistant patients in the Netherlands (Bertina et al, Nature 369:64, 1994), in the USA (Greengard et al, Lancet 343:1361, 1994), and in France (Alhenc-Gelas et al, Lancet 344:555, 1994). The identification of a single point mutation in Factor V which causes a significant proportion of APC resistance in patients contrasts markedly with the large spectrum of different mutations responsible for antithrombin, protein C, or protein S deficiencies (Lane et al, Thromb. Haemost. 70:361, 1993; Reitsma et al, Thromb. Haemost. 69:77, 1993).

The foregoing specification, including the specific embodiments and examples, is intended to be illustrative of the present invention and is not to be taken as limiting. Numerous other variations and modifications can be effected without departing from the true spirit and scope of the invention.

What is claimed is:

1. A human genetic screening method for identifying a mutation in a Factor V gene comprising detecting in a nucleic acid sample isolated from a human the presence of a genetic mutation characterized as a change from a guanine nucleotide to an adenine nucleotide at nucleotide position 205 in exon 10 of the Factor V gene, thereby identifying said mutation.

2. The method according to claim 1, wherein said detecting comprises:
(a) treating, under amplification conditions, a sample of genomic DNA from a human with a polymerase chain reaction (PCR) primer pair for amplifying a region of human genomic DNA containing said nucleotide position 205, said treating producing an amplification product containing said region; and
(b) assaying in the amplification product of step (a) the presence of a change from a guanine nucleotide to an adenine nucleotide at said nucleotide position 205, thereby identifying said mutation.

3. The method according to claim 2 wherein said region contains a nucleotide sequence shown in SEQ ID NO 7, or a fragment thereof.

4. The method according to claim 3 wherein said region consists essentially of a nucleotide sequence shown in SEQ ID NO 3.

5. The method according to claim 2 wherein said region contains a nucleotide sequence shown in SEQ ID NO 23, or a fragment thereof.

6. The method according to claim 5 wherein said region consists essentially of a nucleotide sequence shown in SEQ ID NO 19.

7. The method according to claim 2 wherein said region contains a nucleotide sequence selected from the group consisting of nucleotide sequences shown in SEQ ID NOS 6 and 25, or fragments thereof.

8. The method according to claim 2 wherein said region consists essentially of a nucleotide sequence selected from the group consisting of nucleotide sequences shown in SEQ ID NOS 2 and 18.

9. The method according to claim 2 wherein said PCR primer pair comprises:
(I) a first primer that hybridizes to a noncoding strand of said Factor V gene at a location 3′ to said nucleotide position 205 of said noncoding strand; and
(ii) a second primer that hybridizes to a coding strand of said Factor V gene at a location 3′ to said nucleotide position 205 of said coding strand.

10. The method according to claim 9 wherein said first primer has the nucleotide sequence, 5′-CATACTACAGTGACGTGGAC-3′ (SEQ ID NO 4).

11. The method according to claim 9 wherein said second primer has the nucleotide sequence, 5′-TGTTCTCTTGAAGGAAATGC-3′ (SEQ ID NO 5).

12. The method according to claim 9 wherein said second primer has the nucleotide sequence, 5′-TTACTTCAAGGACAAAATACCTGTAAAGCT-3′ (SEQ ID NO 24).

13. The method according to claim 2 wherein said PCR primer pair produces an amplification product containing a restriction endonuclease site if said mutation is not present, and said assaying of step (b) comprises treating, under restriction conditions, the amplification product of step (a) with a restriction endonuclease that recognizes said site and cleaves said amplification product resulting in restriction products, and detecting the presence of said restriction products.

14. The method according to claim 13 wherein said primer pair comprises a first primer having the nucleotide sequence shown in SEQ ID NO 4 and a second primer having the nucleotide sequence shown in SEQ ID NO 5.

15. The method according to claim 13 wherein said restriction endonuclease is Mnl I and said site has the nucleotide sequence, 5′-ACAGGCGAGG-3′ (SEQ ID NO 6).

16. The method according to claim 2 wherein said PCR primer pair produces an amplification product containing a restriction endonuclease site if said mutation is present, and said assaying of step (b) comprises treating, under restriction conditions, the amplification product of step (a) with a restriction endonuclease that recognizes said site and cleaves said amplification product resulting in restriction products, and detecting the presence of said restriction products.

17. The method according to claim 16 wherein said primer pair comprises a first primer having the nucleotide sequence shown in SEQ ID NO 4 and a second primer having the sequence shown in SEQ ID NO 24.

18. The method according to claim 16 wherein said restriction endonuclease is Hind III and said site has the nucleotide sequence, 5′-AAGCTT-3′ (SEQ ID NO 23).

19. A human genetic screening method for identifying a genetic mutation at nucleotide position 1691 in a Factor V cDNA comprising:
(a) isolating messenger RNA (mRNA) from a human and synthesizing a complementary strand of DNA (cDNA);
(b) treating, under amplification conditions, said cDNA of step (a) with a polymerase chain reaction (PCR) primer pair for amplifying a region of human cDNA containing said nucleotide position 1691 of Factor V cDNA, said treating producing an amplification product containing said region; and
(c) assaying in the amplification product of step (b) the presence of a change from a guanine nucleotide to an adenine nucleotide at said nucleotide position 1691, thereby identifying said mutation.

20. The method according to claim 19 wherein said region contains a nucleotide sequence shown in SEQ ID NO 7, or a fragment thereof.

21. The method according to claim 20 wherein said region consists essentially of a nucleotide sequence selected from the group consisting of sequences shown in SEQ ID NO 26, from nucleotide position 9 to 6917, SEQ ID NO 26, from nucleotide position 1601 to 1724, and SEQ ID NO 28.

22. The method according to claim 19 wherein said region contains a nucleotide sequence shown in SEQ ID NO 6, or a fragment thereof.

23. The method according to claim 22 wherein said region consists essentially of a nucleotide sequence selected from the group consisting of sequences shown in SEQ ID NO 13, from nucleotide position 1601 to 1724, and SEQ ID NO 27.

24. The method according to claim 19 wherein said PCR primer pair comprises:
(I) a first primer that hybridizes to a noncoding strand of said cDNA at a location 3′ to said nucleotide position 1691 of said noncoding strand; and
(ii) a second primer that hybridizes to a coding strand of said cDNA at a location 3′ to said nucleotide position 1691 in said coding strand.

25. The method according to claim 24 wherein said first primer has the nucleotide sequence, 5′-CAGGAAAGGAAGCATGTTCC-3′ (SEQ ID NO 10) or 5′-CATACTACAGTGACGTGGAC-3′ (SEQ ID NO 4).

26. The method according to claim 24 wherein said second primer has the nucleotide sequence, 5′-TGCCATTCTCCAGAGCTAGG-3′ (SEQ ID NO 11) or 5′-TGCTGTTCGATGTCTGCTGC-3′ (SEQ ID NO 12).

27. The method according to claim 19 wherein said amplification product contains a restriction endonuclease site if said mutation is not present, and said assaying of step (c) comprises treating, under restriction conditions, the amplification product of step (b) with a restriction endonuclease that recognizes said site and cleaves said amplification products resulting in restriction products, and detecting the presence of said restriction products.

28. The method according to claim 27 wherein said primer pair comprises a first primer having the nucleotide sequence shown in SEQ ID NO 4 and a second primer having the nucleotide sequence shown in SEQ ID NO 12.

29. The method according to claim 27 wherein said restriction endonuclease is Mnl I and said site has the nucleotide sequence, 5′-ACAGGCGAGG-3′ (SEQ ID NO 6).

30. A diagnostic kit useful for the detection of a genetic mutation in a Factor V gene at nucleotide position 205 of exon 10 associated with activated Protein C resistance in a patient sample, wherein said kit comprises, in an amount sufficient to perform at least one assay, a pair of primers comprising a first primer and a second primer capable of producing by polymerase chain reaction (PCR) an amplification product that contains said nucleotide position 205 of said Factor V gene.

31. The diagnostic kit according to claim 30 wherein said first and second primers are in separate containers.

32. The diagnostic kit according to claim 30 wherein said first primer has the nucleotide sequence shown in SEQ ID NO 4 and said second primer has the nucleotide sequence shown in SEQ ID NO 5.

33. The diagnostic kit according to claim 30 wherein said first primer has the nucleotide sequence shown in SEQ ID NO 4 and said second primer has the nucleotide sequence shown in SEQ ID NO 24.

34. The diagnostic kit according to claim 30 wherein said first primer has the nucleotide sequence shown in SEQ ID NO 10 and said second primer has the nucleotide sequence shown in SEQ ID NO 11.

35. The diagnostic kit according to claim 30 wherein said first primer has the nucleotide sequence shown in SEQ ID NO 4 and said second primer has the nucleotide sequence shown in SEQ ID NO 12.

36. The diagnostic kit according to claim 30 further comprising a control polynucleotide sequence derived from a normal Factor V gene, wherein said sequence is selected from the group of nucleotide sequences shown in SEQ ID NOS 2, 18, 13 from nucleotide position 1601 to 1724, and 27.

37. The diagnostic kit according to claim 30 further comprising a control polynucleotide sequence of claim 43, said sequence derived from a Factor V gene having a genetic mutation at nucleotide position 205 in exon 10.

38. The diagnostic kit according to claim 30 further comprising a control polynucleotide sequence of claim 45, said sequence derived from a Factor V gene having a genetic mutation at nucleotide position 205 in exon 10.

39. The diagnostic kit according to claim 30 further comprising a control polynucleotide sequence of claim 46, said sequence derived from a Factor V gene having a genetic mutation at nucleotide position 205 in exon 10.

40. The diagnostic kit according to claim 30 further comprising a control polynucleotide sequence of claim 47, said sequence derived from a Factor V gene having a genetic mutation at nucleotide position 205 in exon 10.

41. An isolated polynucleotide sequence composition derived from a Factor V gene having a genetic mutation at nucleotide position 205 in exon 10 wherein said sequence comprises a nucleotide sequence from about 40 nucleotides to 6909 nucleotides in length.

42. The polynucleotide sequence according to claim 41 wherein said sequence contains a nucleotide sequence shown in SEQ ID NO 7, or a fragment thereof.

43. The polynucleotide sequence according to claim 41 wherein said sequence consists essentially of a nucleotide sequence shown in SEQ ID NO 3.

44. The polynucleotide sequence according to claim 41 wherein said sequence contains a nucleotide sequence shown in SEQ ID NO 23, or a fragment thereof.

45. The polynucleotide sequence according to claim 141 wherein said sequence consists essentially of a nucleotide sequence shown in SEQ ID NO 19.

46. The polynucleotide sequence according to claim 41 wherein said sequence consists essentially of a nucleotide sequence shown in SEQ ID NO 28.

47. The polynucleotide sequence according to claim 41 wherein said sequence consists essentially of a nucleotide sequence shown in SEQ ID NO 26 from nucleotide position 1601 to 1724.

48. The polynucleotide sequence according to claim 41 wherein said sequence consists essentially of a nucleotide sequence shown in SEQ ID NO 26 from nucleotide position 9 to 6917.

49. A polynucleotide primer comprising a nucleotide sequence shown in SEQ ID NO 24 from nucleotide position 25 to nucleotide position 30 wherein said primer is capable of producing by polymerase chain reaction (PCR) an amplification product containing a Hind III restriction endonuclease site in a Factor V gene having a guanine to adenine point mutation at nucleotide position 205 of exon 10.

50. The polynucleotide primer according to claim 49 consisting essentially of a nucleotide sequence shown in SEQ ID NO 24.


SEQUENCE LISTING (28 sequences)

SEQ ID NO: 1 - DNA (genomic), 11 bp; double-stranded, linear
  misc_difference replace(8, “”): /note= “Wherein N is the nucleotide guanine in the normal allele and the nucleotide adenine in the mutant allele”
gacaggcnagg

SEQ ID NO: 2 - DNA (genomic), 206 bp; double-stranded, linear
catactacagtgacgtggacatcatgagagacatcgcctctgggctaataggactacttctaatctgtaagagcagatccctggacaggcgaggaatacaggtattttgtccttgaagtaacctttcagaaattctgagaatttcttctggctagaacatgttaggtctcctggctaaataatggggcatttccttcaagagaaca

SEQ ID NO: 3 - DNA (genomic), 206 bp; double-stranded, linear
catactacagtgacgtggacatcatgagagacatcgcctctgggctaataggactacttctaatctgtaagagcagatccctggacaggcaaggaatacaggtattttgtccttgaagtaacctttcagaaattctgagaatttcttctggctagaacatgttaggtctcctggctaaataatggggcatttccttcaagagaaca

SEQ ID NO: 4 - DNA (genomic), 20 bp; single-stranded, linear
catactacagtgacgtggac

SEQ ID NO: 5 - DNA (genomic), 20 bp; single-stranded, linear
tgttctcttgaaggaaatgc

SEQ ID NO: 6 - DNA (genomic), 10 bp; single-stranded, linear
acaggcgagg

SEQ ID NO: 7 - DNA (genomic), 11 bp; single-stranded, linear
gacaggcaagg

SEQ ID NO: 8 - DNA (genomic), 20 bp; single-stranded, linear
tgagatcattccaaaggaag

SEQ ID NO: 9 - DNA (genomic), 20 bp; single-stranded, linear
ttgaggtcttaaagagtctc

SEQ ID NO: 10 - DNA (genomic), 20 bp; single-stranded, linear
caggaaaggaagcatgttcc

SEQ ID NO: 11 - DNA (genomic), 20 bp; single-stranded, linear
tgccattctccagagctagg

SEQ ID NO: 12 - DNA (genomic), 20 bp; single-stranded, linear
tgctgttcgatgtctgctgc

SEQ ID NO: 13 - DNA (cDNA), 6925 bp; single-stranded, linear
  mat_peptide 175..6765: /product= “Factor V”
  sig_peptide 91..174
  CDS 91..6765
  misc_feature 1..8
  misc_feature 6918..6925: /standard_name= “EcoRI linker nucleotide sequence”
gaattccgcagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggcactgaattcgtttcttcgaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcagaagtcaagaacatgctaagcgtaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctgggaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttccccagccctcggtcagatgcccctttctccagacctcagccatacaaccctttctctagacttcagccagacaaacctctctccagaactcagccatatgactctctctccagaactcagtcagacaaacctttccccagcccttggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctgtggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattacacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatacaagaaaaaaacggaattc

SEQ ID NO: 14 - protein, 2224 aa [sequence omitted]

SEQ ID NO: 15 - DNA (genomic), 215 bp; double-stranded, linear
gcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacag

SEQ ID NO: 16 - DNA (genomic), 215 bp; double-stranded, linear
gcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcaaggaatacag

SEQ ID NO: 17 - DNA (cDNA), 2297 bp; single-stranded, linear
  misc_difference replace(1614, “”): /label= N /note= “Wherein ”N“ is a guanine in a Factor V normal allele and an adenine in a Factor V mutant allele”
  misc_feature 1614: /note= “Nucleotide position 1614 below corresponds to nucleotide position 1691 of SEQ ID NO 13.”
caggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcnaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggca

SEQ ID NO: 18 - DNA (genomic), 121 bp; double-stranded, linear
catactacagtgacgtggacatcatgagagacatcgcctctgggctaataggactacttctaatctgtaagagcagatccctggacaggcgagctttacaggtattttgtccttgaagtaa

SEQ ID NO: 19 - DNA (genomic), 121 bp; double-stranded, linear
catactacagtgacgtggacatcatgagagacatcgcctctgggctaataggactacttctaatctgtaagagcagatccctggacaggcaagctttacaggtattttgtccttgaagtaa

SEQ ID NO: 20 - DNA (genomic), 11 bp; double-stranded, linear
cctcnnnnnnn

SEQ ID NO: 21 - DNA (genomic), 10 bp; double-stranded, linear
nnnnnngagg

SEQ ID NO: 22 - DNA (genomic), 20 bp; single-stranded, linear
atcgcctctgggctaatagg

SEQ ID NO: 23 - DNA (genomic), 6 bp; double-stranded, linear
aagctt

SEQ ID NO: 24 - DNA (genomic), 30 bp; single-stranded, linear
ttacttcaaggacaaaatacctgtaaagct

SEQ ID NO: 25 - DNA (genomic), 6 bp; double-stranded, linear
gagctt

SEQ ID NO: 26 - DNA (cDNA), 6925 bp; single-stranded, linear
gaattccgcagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcaaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggcactgaattcgtttcttcgaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcagaagtcaagaacatgctaagcgtaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctgggaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttccccagccctcggtcagatgcccctttctccagacctcagccatacaaccctttctctagacttcagccagacaaacctctctccagaactcagccatatgactctctctccagaactcagtcagacaaacctttccccagcccttggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctgtggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattacacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatacaagaaaaaaacggaattc

SEQ ID NO: 27 - DNA (cDNA), 2297 bp; single-stranded, linear
  misc_feature 1614: /note= “Nucleotide position 1614 below corresponds to nucleotide position 1691 of SEQ ID NO 13.”
caggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggca

SEQ ID NO: 28 - DNA (cDNA), 2297 bp; single-stranded, linear
  misc_feature 1614: /note= “Nucleotide position 1614 below corresponds to nucleotide position 1691 of SEQ ID NO 13.”
caggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcaaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaattaggtcattccgaaactcatcattgaaccaggaagaagaagagttcaatcttactgccctagctctggagaatggca